(19) United States
(12) Patent Application Publication
(10) Pub. No.: US 2011/0301131 A1
(43) Pub. Date: Dec. 8, 2011

(54) Drug Delivery Implants and Processes for Their Preparation

(76) Inventors: Joan Fitzpatrick, County Westmeath (IE); David Prior, County Dublin (IE)

(21) Appl. No.: 13/128,057

(22) PCT Filed: Nov. 6, 2009
(86) PCT No.: PCT/IE2009/000078

§ 371 Date: Aug. 24, 2011

(30) Foreign Application Priority Data
Nov. 6, 2008 (IE) S2008/0891

(51) Int. Cl. A61K 31/616; A61K 47/42; A61K 31/167; A61K 31/445; A61K 31/47; A61K 31/485; A61K 31/4468; A61K 31/454; A61K 31/4535; A61K 31/137; A61K 31/196; A61K 31/192; A61K 31/5415; A61K 31/405; C07K 1/107; A61P 23/00; A61P 25/04; A61P 29/00; A61P 31/00; B01J 19/08; C07K 14/78

(52) U.S. Cl. 514/165; 530/356; 514/773; 514/626; 514/330; 514/312; 514/282; 514/329; 514/326; 514/279; 514/646; 514/654; 514/567; 514/570; 514/569; 514/226.5; 514/420; 204/157.63

(57) ABSTRACT

The invention discloses an implant suitable for delivery of at least one drug, the implant comprising a fibrillar collagen matrix having, as measured in Example 1, a viscosity of greater than 100 mPas, optionally greater than 103 mPas, further optionally greater than 106 mPas, still further optionally greater than 109 mPas when a collagen dispersion formed from 140 mg of the fibrillar collagen matrix is dispersed in 25 ml of 2 mM HCl at a pH of less than 3.5 and at a temperature of 30.0+/−0.5° C. The invention also discloses a process for preparing an implant suitable for delivery of at least one drug, the process comprising the steps of forming a fibrillar collagen matrix from a collagen suspension; and carrying out a crosslinking step on either the fibrillar collagen matrix or the collagen suspension under conditions such that the fibrillar collagen matrix has, as measured in Example 1, a viscosity of greater than 100 mPas, optionally greater than 103 mPas, further optionally greater than 106 mPas, still further optionally greater than 109 mPas, when a collagen dispersion formed from 140 mg of the fibrillar collagen matrix is dispersed in 25 ml of 2 mM HCl at a pH of less than 3.5 and at a temperature of 30.0+/−0.5° C. The invention further discloses use of the aforementioned fibrillar collagen matrix for the manufacture of the aforementioned implant for extended local delivery adjacent the site of implantation of at least one drug from the implant.

### FIELD OF THE INVENTION

This invention relates to drug delivery implants and processes for their preparation.

### RELATED BACKGROUND ART

Current efforts in the area of drug delivery include targeted delivery in which the drug is only active in the target area of the body (for example, in cancerous tissues) and sustained release formulations in which the drug is released over a period of time in a controlled manner from the formulation.

Collagens

Collagen is the most abundant protein in the human body and accounts for approximately 30% of all proteins. It comprises about 95% of bone, 75% of skin, and is the main component of other connective tissues (cartilage, tendon and ligament). Collagen naturally biodegrades into component amino acids to be taken up and used by the body in re-building collagen. Collagen surrounds the cells and forms the 3-D cellular matrix of all tissue, giving each its characteristic structure, texture and shape. More than twenty different types of collagen are known and these have been classified according to molecular composition and tissue distribution (Gelse et al. 2003). Bone, dermis, tendon, and ligament are predominantly type I collagen.

The structure, function and biosynthesis of collagens have been thoroughly investigated (Gelse et al. 2003, Nimmi et al. 1988). The type I collagen molecule is made up of three peptide subunits, all having similar amino acid composition and conformed as a triple helix. In addition to the helical portion, the terminal amino acid sequence at each end of the molecule is comprised of short (less than 5% of the total) non-helical domains called telopeptides, which are involved in non-covalent polymerisation with adjacent helices. Subsequent formation of intra- and inter-molecular crosslinks aid in the formation of collagen fibres, fibrils and macroscopic bundles that combine to form tissue.

The main sources of type I collagen for biomedical applications are either animal skin (predominantly bovine or porcine) or Achilles tendon, which is usually of bovine or equine origin. After extraction and purification, the primary to tertiary structure of the collagen may either be preserved (commonly referred to as “insoluble”, “fibrillar” or “native” collagen) or, alternatively, the collagen may be further digested and degraded by enzymes and/or extreme pH leading to partial or complete removal of higher order fibril structures (so-called “soluble” collagen).

Collagen-Derived Medical Products

Collagen is well established as a safe and effective biomaterial. It combines the properties of high tensile strength, biocompatibility and absorbability in living tissue. Collagen-based medical devices are widely used, including haemostats, blood vessel prostheses, heart valves and urinary sphincter implants.

The physiological properties of the soluble and insoluble collagens are different, which has led to a wide variety of medical applications. Soluble collagen can be used to produce biodegradable or non-biodegradable materials with excellent mechanical properties and biocompatibility, whereas insoluble collagen additionally retains the haemostatic (halting of bleeding) and wound healing properties of native collagen.

Surgical Haemostats

The histological and biochemical fate of implanted insoluble collagen has been well studied. Collagen implants first become populated with a number of cell types, primarily those cells responsible for production of fibrous tissue (fibroblasts) (Anselme et al. 1990). It has been found that new collagen production by fibroblasts is increased when the cells are bound to an extracellular matrix, such as a collagen implant (Postlethwaite et al. 1978). The various types of collagen show different susceptibility to collagenolytic degradation. After the triple-helix is cleaved, further degradation of the collagen molecules is facilitated by gelatinases and non-specific proteinases that cleave the primary fragments into small peptides and amino acids, such that the implant is gradually remodelled and replaced by host type I collagen (Burke et al. 1983). This sequence of cellular response, absorption and remodelling of a collagen implant is classic to the normal wound healing mechanism (Cooper, Chapter 7) and studies have demonstrated that such implants accelerate this natural process (Leipziger et al. 1985). Haemostats are commonly used in both surgery and emergency medicine to control bleeding, especially from a torn blood vessel, until the bleeding can be repaired by stitches or other surgical techniques. During the process of blood clotting, platelets become activated by thrombin and aggregate at the site of injury. Stimulated by fibrinogen, the platelets then clump by binding to the collagen that becomes exposed following rupture of the endothelial lining of blood vessels. Haemostatic activity is an inherent property of native collagen and is dependent on the helical structure of the protein. As such, collagen can be a natural haemostat and a wide variety of collagen-based products are used in surgery and dentistry to control excessive bleeding or haemorrhage.

A wide variety of local haemostats have become commercially available, such as gelatin sponge/powder (J&J's Surgifoam®, Pfizer's Gelfoam®); collagen sponge/powder/fibre/sheet; oxidised cellulose (J&J's Surgicel®); thrombin; collagen combined with haemostatic agents such as aprotinin, thrombin and fibrin sealant; gelatin with thrombin and fibrin glues. Proper handling of local haemostats is essential to control bleeding, and for that, the form or configuration of local haemostat is an important factor.

Most collagen based haemostatic agents are referred to as microfibrillar collagen haemostats (MCH), including J&J's Instat MCH ®(although this is not in a sponge format—it's in a fibre format) and Davol's Avitene ®(comes in a powder/flour form and in sheets) and Davol's Ultrafoam ® collagen sponge (Davol claim that the Ultrafoam does not swell. Along with Avitene, UltraFoam is the only collagen haemostat indicated for use in neurosurgery). The surgeon presses the MCH against a bleeding site and the collagen attracts and helps with the clotting process to eventually stop bleeding. Johnson & Johnson's Instat is comprised of purified and lyophilised bovine dermal collagen, prepared as a sponge-like pad, is lightly crosslinked, sterile, non-pyrogenic, and absorbable. J&J's Instat MCH product leaflet suggests that Instat should not be overpacked into cavities or closed spaces as it may absorb fluid and expand and press upon neighbouring structures.

Local haemostats can absorb fluid (body fluid/moisture) and expand pressing nerves in the surrounding tissue against bone or hard tissue (Tomizawa 2005). In a reported case, paraplegia was diagnosed one day after thoracotomy and was caused by spinal cord compression due to Surgicel used in surgery (Iwabuchi et al. 1997). Brodbelt et al 2002 reported cases of paraglegia after thoracotomy surgery due to Surgicel migrating and causing spinal cord compression. Cases of paraplegia after thoracotomy and lumbar laminectomy have been reported and occurred mostly within 24h after surgery (Lovstad et al 1999, Awwad and Smith 1999). Even in an enclosed space, Gelfoam also expanded and compromised the spinal cord (Alander and Stauffer, 1995; Friedman and Whitecloud, 2001).

When an absorbable haemostatic agent is used on or near bony or neural spaces (FDA Public Health Notification, 2004), the Food and Drug Administration (FDA) has recommended that the minimum amount of haemostat necessary to achieve haemostasis be used and that as much of the agent as possible be removed after haemostasis is achieved. Since 1996, FDA has received reports of over 110 adverse events related to absorbable haemostatic agents. Eleven of the events resulted in paralysis or other neural deficits. The last reported paralysis occurred in October, 2003. The common thread in all 11 events was an absorbable haemostatic agent that was used on or near a bony or neural space and left inside the patient. When wetted, the material swelled and exerted pressure on the spinal cord or other neural structures, resulting in pain, numbness or paralysis.

Collagen-Based Localised Drug Delivery Systems

The concept of delivering a drug directly to a specific tissue, organ or region of intended action is receiving increasing attention in the medical community (Patterson et al. 2003). The key benefit of localised drug delivery over systemic therapy is that high concentrations of drug can be maintained at the target site, while avoiding risk of systemic toxicity and associated side effects. Many of today's products aimed at localised delivery are device-drug combinations. Examples include pumps for continuous infusion of local anaesthetic, medicated stents and bone cements containing an antibiotic.

It is an object of the invention to provide a drug delivery implant that obviates some of the aforementioned problems associated with the prior art.

### SUMMARY OF THE INVENTION

The present invention is directed to an implant for drug delivery and a process for its preparation.

In a first aspect of the invention, the invention is directed to an implant suitable for delivery of at least one drug, the implant comprising a fibrillar collagen matrix having, as measured in Example 1, a viscosity of greater than 100 mPas, optionally greater than 103 mPas, further optionally greater than 106 mPas, still further optionally greater than 109 mPas when a collagen dispersion formed from 140 mg of the fibrillar collagen matrix is dispersed in 25 ml of 2 mM HCl at a pH of less than 3.5 and at a temperature of 30.0+/−0.5° C. By “30.0+/−0.5° C.” is meant a temperature within the range of 29.5 to 30.5° C. It will, of course, be appreciated, by reference to Example 1 itself, that, if one or more drugs are dispersed in the fibrillar collagen matrix, then the viscosity values given above refer to the collagen dispersion itself before any drug(s) is/are dispersed herein and not to the drug delivery device comprising at least one drug dispersed in the fibrillar collagen matrix. Optionally, the viscosity of the sterile fibrillar collagen matrix is at least 70% of the viscosity of the non-sterile fibrillar collagen matrix.

In a second aspect of the invention, the invention is directed to a process for preparing an implant suitable for delivery of at least one drug, the process comprising the steps of
(i) forming a fibrillar collagen matrix from a collagen suspension; and
(ii) carrying out a crosslinking step on either the fibrillar collagen matrix or the collagen suspension under conditions such that the fibrillar collagen matrix has, as measured in Example 1, a viscosity of greater than 100 mPas, optionally greater than 103 mPas, further optionally greater than 106 mPas, still further optionally greater than 109 mPas, when a collagen dispersion formed from 140 mg of the fibrillar collagen matrix is dispersed in 25 ml of 2 mM HCl at a pH of less than 3.5 and at a temperature of 30.0+/−0.5° C.

It will, of course, be appreciated, by reference to Example 1 itself, that, if one or more drugs are dispersed in the fibrillar collagen matrix, then the viscosity values given above refer to the collagen dispersion itself before any drug(s) are dispersed herein and not to the drug delivery device comprising at least one drug dispersed in the fibrillar collagen matrix. The inventors have noted that crosslinking either the collagen suspension or the fibrillar collagen matrix, before or after incorporation of the at least one drug, whilst ensuring that the collagen dispersion, as measured in Example 1, has a viscosity of greater than 100 mPas, optionally greater than 103 mPas, further optionally greater than 106 mPas, still further optionally greater than 109 mPas, is unexpectedly is associated with an extended clinical efficacy for drug delivery from the implant.

Optionally, the crosslinking step is carried out on the collagen fibrillar matrix, before or after incorporation of the at least one drug into the fibrillar collagen matrix. Further optionally, the crosslinking step is carried out on the fibrillar collagen matrix after incorporation of the at least one drug into the fibrillar collagen matrix.

In a further aspect of the invention, there is provided use of a fibrillar collagen matrix having, as measured in Example 1, a viscosity of greater than 100 mPas, optionally greater than 103 mPas, further optionally greater than 106 mPas, still further optionally greater than 109 mPas when a collagen dispersion formed from 140 mg of the fibrillar collagen matrix is dispersed in 25 ml of 2 mM HCl at a pH of less than 3.5 and at a temperature of 30.0+/−0.5° C. for the manufacture of the implant of the first aspect of the invention for extended local delivery adjacent to the site of implantation of at least one drug from the implant. As will be observed from FIG. 7, serum levels of a drug can no longer be detected 24 hours after implantation of an implant comprising a fibrillar collagen matrix sterilised by gamma radiation alone and not forming part of this invention. In contrast, as will also be observed from FIG. 7, serum levels of a drug can still be detected more than 42 hours after implantation of an implant comprising a fibrillar collagen matrix sterilised by EO sterilisation. It will be appreciated, therefore, drug delivery from an implant of the present invention has a duration at least 50% longer, optionally at least 75% longer, than that of an implant not of the invention.

The in-vivo release profile of certain drugs from the implant of the present invention can be observed through pharmacokinetic (PK) assessments in both animals and humans. The PK profile of such systems indicates, surprisingly, a double peak in serum concentration. One possible explanation for this is that, as the crystalline drug in the fibrillar collagen matrix dissolves, the structure of fibrillar collagen matrix collapses yielding the initial release of drug and the associated first peak in the serum PK profile. It is thought that the second phase of drug release from the collapsed fibrillar collagen matrix may be due to the reduction in porosity and formation of a hydrogel-type material, which affords the second PK peak.

FIGS. 6a, 7, 9 and 12 discussed hereunder demonstrate that carrying out the crosslinking step using EO sterilisation or E-beam sterilisation, but not by gamma irradiation alone is, surprisingly, associated with an extended clinical efficacy for drug delivery from the implant in both beagle dogs and humans. It will, of course, be appreciated that such an extended clinical efficacy can be desirable. It will also be appreciated that such an extended clinical efficacy can be expected by the implantation of the invention in any animal.

The invention also discloses a sterile drug delivery implant comprising a fibrillar collagen matrix having a relative viscosity that is at least similar to the relative viscosity of the fibrillar collagen matrix of a non-sterile drug delivery implant, each at a pH 4.5 and 37° C. Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix. Still further optionally, the relative viscosity of the sterile fibrillar collagen matrix is within the range of about 90% of the relative viscosity of the non-sterile fibrillar collagen matrix to about 5 times the relative viscosity of the non-sterile fibrillar collagen matrix, optionally within the range of about 90% of the relative viscosity of the non-sterile fibrillar collagen matrix to about 3 times the relative viscosity of the non-sterile fibrillar collagen matrix.

By the term “sterile”, we mean free of living germs and microorganisms. Methods of terminal sterilisation include treatment with heat, ethylene oxide or radiation such as e-beam or gamma rays. All of these methods of terminal sterilisation typically result in increased crosslinking with proteins such as collagen. In addition, terminal sterilisation by treatment with heat, or radiation such as e-beam or gamma rays typically results in some level of scission or breaking of molecular bonds, especially in the case of proteins such as collagen.

Optionally, the invention discloses a sterile drug delivery implant comprising a fibrillar collagen matrix having a relative viscosity measured in an Ostwald viscometer, when the fibrillar collagen matrix is dispersed to homogeneity at a concentration of 0.56 g in 100 ml deionised water at pH 4.5 and 37° C., of greater than 1.5. Further optionally, the relative viscosity is greater than 1.7. Further optionally, the relative viscosity is greater than 2.5. Alternatively, the invention is directed to an implant for drug delivery, the implant comprising a fibrillar collagen matrix having a relative viscosity in a Brookfield viscometer that is at least similar to the relative viscosity of the fibrillar collagen matrix of a non-sterile drug delivery implant, each at a pH 4.5 and 37° C. Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix.

Preferably, a pharmaceutically acceptable amount of at least one drug is dispersed in the fibrillar collagen matrix.

Optionally, the drug delivery implant of the present invention shows a volume reduction of at least 30%, when 70mg of the fibrillar collagen matrix is immersed in 50 ml saline (solution of 0.9% sodium chloride) for 10 minutes at 37° C. Optionally, the volume reduction is at least 50%. Further optionally, the volume reduction is at least 70%. Without being bound by theory, it is thought that a viscosity of greater than 100 mPas, as measured in Example 1, for a collagen dispersion formed from a fibrillar collagen matrix provides structure to the gel formed when the fibrillate collagen matrix is contacted, in vivo, with an aqueous-based medium (bodily fluids, for example) and forms a hydrogel. Without being bound by theory, it is thought that the fibrillar collagen matrix relative viscosity of greater than 1.5 (as measured in an Ostwald viscometer, as described above) provides structure to the gel formed when the matrix is contacted, in vivo, with an aqueous-based medium and forms a hydrogel.

It will be appreciated that a non-sterile fibrillar collagen matrix cannot be used in the human or animal body or on an open wound of a human or animal body. It is the aim of the present invention to provide a drug delivery implant that has a relative viscosity that is at least similar to the non-sterile fibrillar collagen matrix. This relative viscosity can be achieved by selecting those forms of sterilisation that, as well as causing some level of molecular damage (e.g. scission or breaking of some crosslinks resulting from irradiation), promote crosslinking and either restore the degree of crosslinking to that observed in the native unsterile fibrillar collagen matrix or even enhance the degree of crosslinking beyond that observed in the native non-sterile fibrillar collagen matrix.

Alternatively, or additionally, the fibrillar collagen matrix can be sterilised using gamma irradiation (which can create crosslinks in synthetic polymers but the level of scission damage is greater and so is not compensated by the radiation crosslinks) wherein the fibrillar collagen matrix has already been treated by dehydrothermal crosslinking or chemical crosslinking, or both, before the gamma irradiation.

The optional volume reduction of at least 30% is obtainable by lyophilising the collagen suspension and preparing a collagen sponge used in the implants of the present invention at a concentration of the polymer of below 25 mg/ml (for example, in the range of 2.5 to 11.2 mg/ml, optionally about 5.6 mg/ml) at a pH of less than 4.9, for example between 3.6 and 4.9. A concentration of less than 11.2 mg/ml, for example about 5.6 mg/ml, was selected based on viscosity and handling of the collagen suspension, flow properties of the collagen suspension, ease of dispensing into moulds and subsequent sponge characteristics. The pH was chosen to achieve a preferred pH of about 4.5 before any drug is added to the collagen suspension (in situations where the addition of the drug causes a change in pH, the pH of the collagen suspension may be adjusted immediately prior to drug addition in order to compensate for such change).

For the pH, the upper limit of the collagen suspension (whether the drug is present or is not) should be set at pH 4.9. pH 4.9 has been empirically chosen so that the collagen suspension is sufficiently acidic to allow for swelling of the collagen fibres to reduce the viscosity to allow for suspension homogeneity and facilitate further processing, such as pumping into moulds, etc. For example, when the drug is bupivacaine hydrochloride, the final pH of the collagen suspension, after the addition of the drug, is ideally pH 3.9±0.3, although, before the drug addition, it is kept at about pH 4.5.

In a second aspect of the invention, the invention discloses a drug delivery implant showing a volume reduction of least 30% when 70 mg of the fibrillar collagen matrix, is immersed in 50 ml saline for 10 minutes at 37° C. Optionally, the volume reduction is at least 50%. Further optionally, the volume reduction is at least 70%.

Optionally, the fibrillar collagen matrix has a relative viscosity at pH 4.5 and 37° C., when the matrix is dispersed at a concentration of 0.56 g in 100 ml deionised water, of greater than 1.5 (when measured in an Ostwald viscometer, as described above). Further optionally, the relative viscosity is greater than 1.7. Still further optionally, the relative viscosity is greater than 2.5. Preferably, a pharmaceutically relevant amount of at least one drug is dispersed in the fibrillar collagen matrix. It will, of course, be appreciated that, if one or more drugs are dispersed in the fibrillar collagen matrix, then the relative viscosity values given above refer to the fibrillar collagen matrix itself before any drug(s) are dispersed herein and not to the drug delivery device comprising at least one drug dispersed in the fibrillar collagen matrix.

In a further aspect of the invention, there is disclosed a process for preparing a sterile drug delivery implant according to the first aspect of the invention, the process comprising crosslinking the fibrillar collagen matrix to a relative viscosity that is at least similar to the relative viscosity of a non-sterile drug delivery implant, each at pH 4.5 and 37° C.

Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix. Still further optionally, the relative viscosity of the sterile fibrillar collagen matrix is within the range of about 90% of the relative viscosity of the non-sterile fibrillar collagen matrix to about 5 times the relative viscosity of the non-sterile fibrillar collagen matrix, optionally within the range of about 90% of the relative viscosity of the non-sterile fibrillar collagen matrix to about 3 times the relative viscosity of the non-sterile fibrillar collagen matrix.

Optionally, the crosslinking step results in a relative viscosity measured in an Ostwald viscometer, when the fibrillar collagen matrix is dispersed to homogeneity at a concentration of 0.56 g in 100 ml deionised water at pH 4.5 and 37° C. of greater than 1.5. Further optionally, the relative viscosity is greater than 1.7. Further optionally, the relative viscosity is greater than 2.5.

Alternatively, the crosslinking step results in a relative viscosity in a Brookfield viscometer that is at least similar to the relative viscosity of the fibrillar collagen matrix of a non-sterile drug delivery implant. Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix.

The crosslinking step can be ethylene oxide (EO) sterilisation, electron beam (E-beam) sterilisation, dehydrothermal crosslinking, chemical crosslinking, or a combination thereof. Optionally, the crosslinking step can be ethylene oxide (EO) sterilisation or electron beam (E-beam) sterilisation. Without being bound by theory, it is postulated that the increased relative viscosity is caused by increased crosslinking, so that any suitable crosslinking agents should achieve the same end result.

In a further aspect of the invention, there is provided a process for preparing a drug delivery implant according to the second aspect of the invention, the process comprising preparing the fibrillar collagen suspension at a concentration of less than 25 mg/ml, optionally, in a concentration range of 2.5 to 11.2 mg/ml and at a pH of less than 4.9, optionally in the range of 3.6 to 4.9.

### BRIEF DESCRIPTION OF THE FIGURES

FIG. 1 illustrates a drug delivery implant in the form of a sponge.

FIG. 2 illustrates DSC scans comparing different sponge drug delivery implants, which were tested in duplicate, where blue (with open squares) is gamma sterilised collagen sponges, green (with closed circles) is non-sterile collagen sponges and red (with crosses) is EO sterilised collagen sponges.

FIG. 3 is graphical representations illustrating the increase in weight over time for collagen sponges in an aqueous medium (black diamonds is non-sterile collagen; pink squares is ETO (or EO) sterilised collagen; and yellow triangles is gamma sterilised collagen).

FIG. 4a shows a bupivacaine-containing drug delivery implant (such as is prepared according to Example 3).

FIG. 4b is a graphical representation of an Ostwald viscometer used in the study.

FIG. 5 illustrates the reduction in volume of a bupivacaine-containing drug delivery implant.

FIG. 6a illustrates the mean serum PK profile of bupivacaine-containing drug delivery implant in beagle dogs.

FIG. 6b illustrates the individual serum PK profile of bupivacaine-containing drug delivery implant in beagle dogs.

FIG. 7 illustrates PK profiles for EO (blue open squares) and Gamma (red open circles) sterilised bupivacaine-containing drug delivery implants in beagle dogs.

FIG. 8 illustrates a representation of the chain scission that occurs in collagen.

FIG. 9 illustrates mean (and point standard deviations) PK profile of serum bupivacaine in women who underwent hysterectomy.

FIG. 10 illustrates individual PK profiles of serum bupivacaine in 12 women who underwent hysterectomy.

FIG. 11 is an SEM of a bupivacaine—containing implant of the invention, illustrating the microstructure of the fibrillar collagen matrix.

FIG. 12 illustrates mean serum PK Profiles of EO and E-beam sterilised bupivacaine-containing drug delivery implants in beagle dogs (pink closed squares and black closed diamonds each represent EO collagen, and red closed triangles represent e-beam collagen).

### DETAILED DESCRIPTION OF THE INVENTION

The present invention is directed to a drug delivery implant and a process for its preparation.

In a first aspect of the invention, the invention is directed to an implant suitable for delivery of at least one drug, the implant comprising a fibrillar collagen matrix having, as measured in Example 1, a viscosity of greater than 100 mPas, optionally greater than 103 mPas, further optionally greater than 106 mPas, still further optionally greater than 109 mPas, when a collagen dispersion formed from 140 mg of the fibrillar collagen matrix is dispersed in 25 ml of 2 mM HCl at a pH of less than 3.5 and at a temperature of 30.0+/−0.5° C.

In a second aspect of the invention, the invention is directed to a process for preparing an implant suitable for delivery of at least one drug, the process comprising the steps of
(i) forming a fibrillar collagen matrix from a collagen suspension; and
(ii) carrying out a crosslinking step on either the fibrillar collagen matrix or the collagen suspension under conditions such that the fibrillar collagen matrix has, as measured in Example 1, a viscosity of greater than 100 mPas, optionally greater than 103 mPas, further optionally greater than 106 mPas, still further optionally greater than 109 mPas, when a collagen dispersion formed from 140 mg of the fibrillar collagen matrix is dispersed in 25 ml of 2 mM HCl at a pH of less than 3.5 and at a temperature of 30.0+/−0.5° C.

The invention also discloses a sterile drug delivery implant comprising a fibrillar collagen matrix, having a relative viscosity that is at least similar to the relative viscosity of the fibrillar collagen matrix of a non-sterile drug delivery implant. Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix. Still further optionally, the relative viscosity of the sterile fibrillar collagen matrix is within the range of about 90% of the relative viscosity of the non-sterile fibrillar collagen matrix to about 5 times the relative viscosity of the non-sterile fibrillar collagen matrix, optionally within the range of about 90% of the relative viscosity of the non-sterile fibrillar collagen matrix to about 3 times the relative viscosity of the non-sterile fibrillar collagen matrix.

Optionally, the invention discloses a sterile drug delivery implant comprising a fibrillar collagen matrix having a relative viscosity measured in an Ostwald viscometer, when the fibrillar collagen matrix is dispersed to homogeneity at a concentration of 0.56 g in 100 ml deionised water at pH 4.5 and 37° C. of greater than 1.5. Further optionally, the relative viscosity is greater than 1.7. Further optionally, the relative viscosity is greater than 2.5. Alternatively, the invention discloses a sterile drug delivery implant comprising a fibrillar collagen matrix having a relative viscosity in a Brookfield viscometer that is at least similar to the relative viscosity of the fibrillar collagen matrix of a non-sterile drug delivery implant, each at pH 4.5 and 37° C. Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix. More specifically, such viscosity can be measured using the Brookfield Digital Rheometer DV-III+ with circulating bath and Rheocalc software, as follows. For a single point measurement, select the appropriate spindle for the sample to be tested. Attach the spindle to the rheometer and place the bubble-free sample in the heating chamber. Set the temperature at which the sample will be tested and let the sample equilibrate to that temperature. Set the spindle speed to attain the required shear force and verify that the torque is between 10% and 100% to achieve good accuracy. If this torque level is not reached, the speed and or the spindle should be modified. Once this is reached, the viscosity can be read on the visual display unit of the rheometer, or a single point measurement program can be started. An average of 3 viscosity values should be taken.

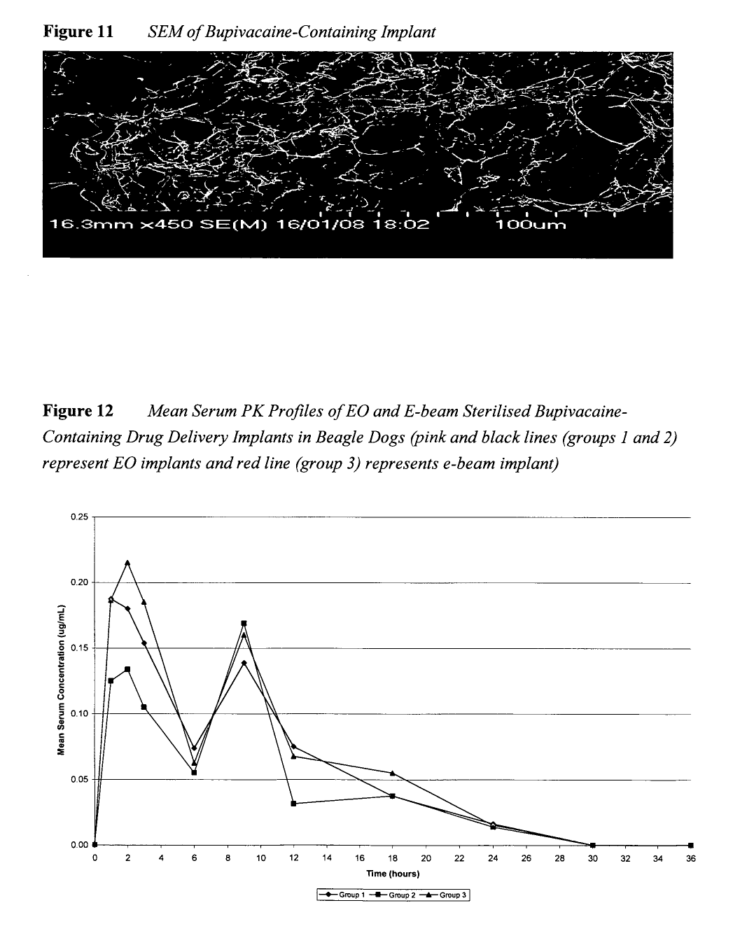

Preferably, a pharmaceutically acceptable amount of at least one drug is dispersed in the fibrillar collagen matrix. It will, of course, be appreciated that, if one or more drugs are dispersed in the fibrillar collagen matrix, then the relative viscosity values given above and the viscosity values given above and measured according to Example 1, refer to the fibrillar collagen matrix itself before any drug(s) are dispersed therein and not to the drug delivery implant comprising at least one drug dispersed in the fibrillar collagen matrix. Optionally, the drug delivery implant also shows a volume reduction of at least 30% when an implant is immersed in excess saline for 10 minutes at 37° C. Optionally, the volume reduction is at least 50%. Further optionally, the volume reduction is at least 70%. Without being bound by theory, it is thought that the primary influence on implant collapse is the microstructure within the fibrillar collagen matrix. This is controlled by the fibrillar nature of the collagen and the concentration and pH of the collagen suspension, from which the fibrillar collagen matrix is formed. The structure of the present drug delivery implant after purification is a combination of fibrils and microfibrils and it is thought that it is this fibrous nature that influences the microstructure created in the implant after lyophilisation. FIG. 11 illustrates the microstructure of a bupivacaine-containing implant of the invention.

For the suspension to be used to prepare a drug delivery implant, collagen concentrations of up to 11.2 mg/ml are used. For the pH, the upper limit of the collagen suspension (whether the drug is present or is not) should be set at pH 4.9. pH 4.9 has been empirically chosen so that the suspension is sufficiently acidic to allow for swelling of the collagen fibres and thereby reduce the viscosity to facilitate suspension homogenisation and facilitate downstream processing, such as pumping into the moulds. For example, when the drug is bupivacaine hydrochloride, the final pH of the collagen suspension, after the addition of the drug, is ideally pH 3.9±0.3, although before the drug addition the target pH is 4.5.

In a further aspect of the invention, there is provided a process for preparing a drug delivery implant according to the first aspect of the invention, the process comprising crosslinking the fibrillar collagen matrix and sterilising the drug delivery implant. The crosslinking and sterilisation steps can be simultaneously achieved by selecting those forms of sterilisation that, as well as causing some level of molecular damage (e.g. scission resulting from radiation), promote crosslinking and either restore the degree of crosslinking to that observed in the native unsterile fibrillar collagen matrix or even enhance the degree of crosslinking beyond that observed in the native unsterile fibrillar collagen matrix. The crosslinking and sterilisation steps can be simultaneously achieved by selecting those forms of sterilisation that, as well as causing some level of molecular damage (e.g. scission resulting from radiation), promote crosslinking and then carrying out those forms of sterilisation under conditions such that the fibrillar collagen matrix has, as measured in Example 1, a viscosity of greater than 100 mPas, optionally greater than 103 mPas, further optionally greater than 106 mPas, still further optionally greater than 109 mPas, when a collagen dispersion formed from 140 mg of the fibrillar collagen matrix is dispersed in 25 ml of 2 mM HCl at a pH of less than 3.5 and at a temperature of 30.0+/−0.5° C. Alternatively, or additionally, the fibrillar collagen matrix can be sterilised using e-beam sterilisation or by gamma irradiation (at least the latter of which can create crosslinks in synthetic polymers but the level of scission damage is greater and so is not compensated by the radiation crosslinks) and can be separately crosslinked by treating with dehydrothermal crosslinking or chemical crosslinking, or both, before, during or after e-beam sterilisation or gamma irradiation.

In a still further aspect of the invention, there is disclosed a process for preparing a drug delivery implant according to the first aspect of the invention, the process comprising crosslinking the fibrillar collagen matrix to a relative viscosity that is at least similar to the relative viscosity of a non-sterile drug delivery implant, each at pH 4.5 and 37° C.

Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix. Still further optionally, the relative viscosity of the sterile fibrillar collagen matrix is within the range of about 90% of the relative viscosity of the non-sterile fibrillar collagen matrix to about 5 times the relative viscosity of the non-sterile fibrillar collagen matrix, optionally within the range of about 90% of the relative viscosity of the non-sterile fibrillar collagen matrix to about 3 times the relative viscosity of the non-sterile fibrillar collagen matrix.

Optionally, the crosslinking step results in a relative viscosity measured in an Ostwald viscometer, when the fibrillar collagen matrix is dispersed to homogeneity at a concentration of 0.56 g in 100 ml deionised water at pH 4.5 and 37° C. of greater than 1.5. Further optionally, the relative viscosity is greater than 1.7. Further optionally, the relative viscosity is greater than 2.5.

Alternatively, the crosslinking step results in a relative viscosity in a Brookfield viscometer that is at least similar to the relative viscosity of the fibrillar collagen matrix of a non-sterile drug delivery implant. Optionally, the relative viscosity of the sterile fibrillar collagen matrix is at least 90% of the relative viscosity of the non-sterile fibrillar collagen matrix. Further optionally, the relative viscosity of the sterile fibrillar collagen matrix is at least 95% of the relative viscosity of the non-sterile fibrillar collagen matrix. Still further optionally, the relative viscosity of the sterile fibrillar collagen matrix is more than the relative viscosity of the non-sterile fibrillar collagen matrix.

The crosslinking step can be ethylene oxide (EO) sterilisation, electron beam (E-beam) sterilisation, dehydrothermal crosslinking, chemical crosslinking, or a combination thereof. Without being bound by theory, it is postulated that the increased relative viscosity is caused by increased crosslinking, so that any suitable crosslinking agents, subject to modification, should achieve the same end result.

In a still further aspect of the invention, there is disclosed a process for preparing a drug delivery implant according to the second aspect of the invention, the process comprising preparing the fibrillar collagen suspension at a concentration of less than 25 mg/ml, optionally, in a concentration range of 2.5 to 11.2 mg/ml and at a pH of less than 4.9, optionally in the range of 3.6 to 4.9.

In a further aspect of the invention, the invention is directed to a drug delivery implant showing a volume reduction of least 30%, when 70 mg of the fibrillar collagen matrix, is immersed in 50 ml saline for 10 minutes at 37° C. Optionally, the volume reduction is at least 50%. Further optionally, the volume reduction is at least 70%. In a still further aspect of the invention, there is provided a process for preparing a drug delivery implant showing a volume reduction of least 30%, when 70 mg of the fibrillar collagen matrix, is immersed in 50 ml saline for 10 minutes at 37° C., the process comprising preparing the fibrillar collagen suspension at a concentration of less than 25 mg/ml, optionally in a concentration range of 2.5 to 11.2 mg/ml and at a pH of less than 4.9, optionally in the range of 3.6 to 4.9.

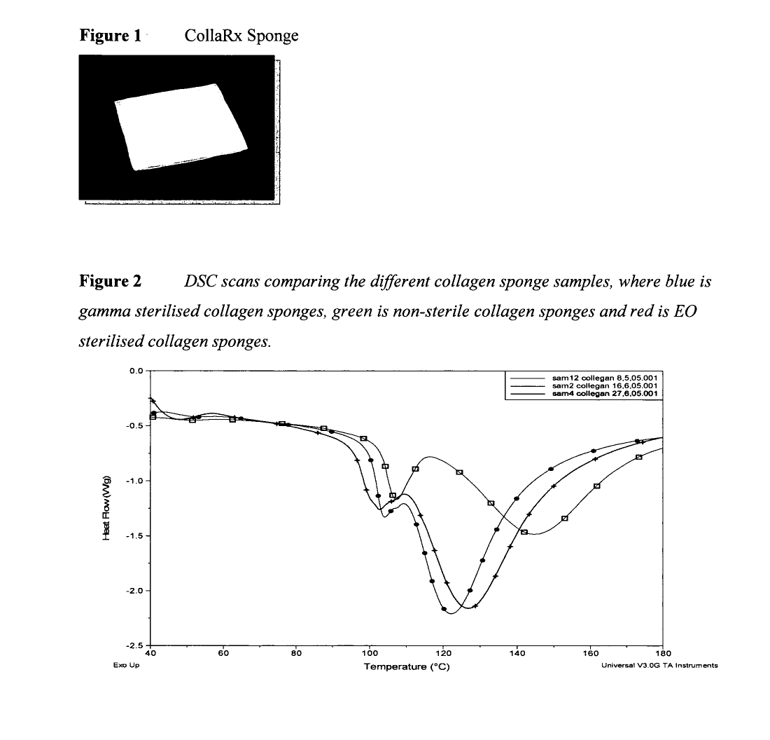

Biodegradable polymers make ideal vehicles for localised drug delivery. Collagen offers the advantages of a natural and well-established biocompatible material, together with its complimentary wound healing and haemostatic properties. This technology uses collagen for localised drug delivery, based upon a type I collagen matrix derived from bovine or equine Achilles tendon. The drug delivery implants of the first aspect of the invention may be provided as sponges. More specifically, the drug delivery implants of the first aspect of the invention may be formatted as a lyophilised porous sponge (FIG. 1). In vivo, drug is released by a combination of diffusion and natural enzymatic breakdown of the fibrillar collagen matrix. The fibrillar collagen matrix itself is fully resorbed within one to seven weeks according to implant location (i.e. well vascularised areas versus bone cavities).

The implant is used as a drug delivery system for localised pharmacological action. A wide variety of drugs are known to act locally, including antibacterials, anaesthetics, analgesics and anti-inflammatories, all providing great potential for utilisation of the technology (either as single active or combination active products).

The drug delivery implant has been developed based on a fibrillar collagen matrix that retains the triple helical structure of the collagen molecule and is ideal as a drug delivery system. This is because, unlike other collagen sponges, this porous collagen structure collapses as soon as it becomes wet, thus preventing any expansion and compression of surrounding tissues/nerves. One of the key features of this drug delivery implant is the fact that the collagen matrix is a leave-behind implant that does not put pressure on surrounding tissues/nerves as the matrix collapses and does not expand and is thus ideal for localised drug delivery.

Another key feature of this technology is the in-vivo release profile of certain drugs from the drug delivery device of the present invention, observed through pharmacokinetic (PK) assessments in both animals and humans. The PK profile of such systems indicates, surprisingly, a double peak in serum concentration. One possible explanation for this is as follows; the crystalline drug in the sponge matrix dissolves, facilitating collapse of the collagen matrix structure and yielding the initial release of drug and the associated first peak in the serum PK profile. The second phase of drug release is slower from the collapsed sponge matrix. It is thought that this may be due to the reduction in porosity and formation of a hydrogel-type material, which affords the second PK peak. This double PK peak phenomenon is associated with an extended clinical efficacy for the drug.

One influence on the formation of the hydrogel-type material by absorption of fluid by the collagen sponge matrix is the level of crosslinking in the sponge matrix. This can be increased, by, for example ethylene oxide (EO) sterilisation or electron beam (E-beam) sterilisation. EO sterilisation and E-beam sterilisation are thought to induce crosslinking in the collagen molecule (EO sterilisation, Friess 1998) and this is thought to lead to a higher density and viscosity layer on absorption of fluid by the collagen sponge. This subsequently has an influence on drug release from the collapsed sponge and thus accounts for the second PK peak.

Method A: Crosslinking by Ethylene Oxide (EO) Sterilisation

EO sterilisation plays an important role in the healthcare industry. EO is a potent, anti-microbial agent that can kill all known viruses, bacteria and fungi, annihilating even the most sterilisation-resistant types of microorganisms, bacterial spores. EO is a small molecule in which two carbon and four hydrogen atoms are joined to one oxygen atom in a highly strained ring. Because of the chemical's very low boiling point (10.4° C.), it becomes active at room temperature. It vaporises and permeates rapidly through packaging and dissolving in substances like plastic and rubber. EO readily kills all types of microorganisms under ordinary atmospheric conditions. Its fragile molecular bonds allow it to quickly react with a wide variety of compounds. The resulting chemical reaction is called alkylation.

Effective sterilisation relies on a number of process variables during the sterilisation cycle including (1) a sufficient dose of EO must be used for an adequate length of time to kill the most resistant microorganisms; (2) adequate humidity must be present to facilitate the process and (3) the dose of EO required depends on the temperature of the process (the higher the temperature, the lower the dose of EO necessary to sterilise.)

In order to protect the collagen polymer and to maintain the physical structure of the matrix (especially in the case of the lyophilised sponge), the use of product (drug delivery implant) temperatures above 40° C.-42° C. are to be avoided. One suitable EO steriliser is Type 15009 VD steriliser manufactured by DMB Wiesbaden with a chamber volume of this steriliser is about 15001, but other models and chamber sizes can be used.

Suitable EO sterilisation conditions for use in the sterilisation step of the process of the present invention comprise evacuation down to between −0.07 and −0.8 bar, following which the chamber is charged with sterilising gas up to between +0.4 and +4 bar for 4-6 hours at 30°-60° C. Desorption is effected by alternating +0.6 −1.0 bar overpressure and −0.06-0.8 bar under-pressure on a 10-30 minute cycle for not less than 4 hours.

Other cycles that can be used for the product involve steam injections to enhance the sterilisation process efficiency with secondary evacuation down to −0.268 bar, followed by EO gas injection at incremental pressure up to 0.385 bar at a temperature of 30-60° C. for 4 hours exposure. Desorption is effected by cycling between vacuum pressure of 0.068 bar to a pressurised wash with Nitrogen or air at 0.813 bar to atmospheric pressure at the end of the cycle. It will be appreciated that the temperature and pressure of the EO sterilisation have different functions—temperature enhances the EO reaction and, therefore, efficiency and pressure is used to initially push the EO into the product and later to draw the gas out at the end of the cycle under vacuum.

Method B: Crosslinking by Dehydrothermal and Chemical Crosslinking Processes

Another means of creating crosslinks in collagen material includes dehydrothermal and chemical crosslinking processes. Crosslinking is defined as the establishment of chemical links between the molecular chains in polymers. Collagen fibres can be crosslinked by severe dehydration (dehydrothermal crosslinking) at elevated temperatures and by the use of crosslinking agents including glutaraldehyde, carbodiimides and organic peroxides. The crosslink initiated by glutaraldehyde occurs by the reaction of the glutaraldehyde aldehyde group with two a-amine groups of either lysine or hydroxylysine. Two amine groups are used in every glutaraldehyde-induced primary amine crosslink.

Dehydrothermal (DHT) drying is a physical method of crosslinking that exposes the collagen fibrils to heat at low pressure, driving off residual water molecules. It is believed that these drying conditions initiate the formation of a lysinoalanine amino acid, which forms an amino acid link and is initiated by a hydroxyl group, perhaps from the numerous hydroxyproline residues of the collagen molecule.

Chemical crosslinking agents can be divided into two groups. If the two reactive ends are identical, they are called homobifunctional and those agents with two dissimilar reactive ends are called heterobifunctional. Homobifunctional crosslinkers (including glutaraldehyde, which is an amine-reactive homobifunctional crosslinker) are used in one step reactions while the heterobifunctional crosslinkers are used in two-step sequential reactions, wherein the least labile reactive end is reacted first. Homobifunctional crosslinking agents have the tendency to result in self-conjugation, polymerisation, and intracellular crosslinking. On the other hand, heterobifunctional agents allow more controlled two step sequential reaction which minimises undesirable intra-molecular cross reaction and polymerisation.

The most widely used heterobifunctionals are those with an amine-reactive NHS-ester at one end and sulfhydryl reactive group at the other end. Since the NHS-ester is the least stable in aqueous medium, it is reacted first. After removing unreacted agents, the reaction with the second reactive group is allowed to proceed. The sulfhydryl reactive groups are generally maleimides, pyridyl disulfides and α-haloacetyls. Other widely used crosslinkers are carbodiimides that facilitate reaction between carboxylates (—COOH) and primary amines (—NH₂). There are also heterobifunctional crosslinkers with one photoreactive end.

Method C: Crosslinking by E-Beam Radiation Sterilisation

E-beam irradiation involves an electron accelerator to expose a material to a stream of highly energised electrons or β-particles. The electron beam linear accelerator works similarly to a television tube. Instead of electrons being widely dispersed and hitting a phosphorescent screen at low energy levels, they are concentrated and accelerated close to the speed of light. This produces very quick reactions on molecules in the product being irradiated. E-beam or beta radiation is used in the polymer and medical device industries to create crosslinks to strengthen polymeric materials. E-beam crosslinking technology uses irradiation to link together groups of polymers. The radiation causes binding to occur at multiple sites along the polymer chains. The result of crosslinking is greater tensile and impact strength, increased durability and resistance to deformation, superior solvent and chemical resistance, and greater resistance to abrasion and stress fractures. E-beam radiation of naturally occurring polymers is generally reported as resulting in degradation of the polymer, however, E-beam is being developed for use as a method for crosslinking hydrogels produced from natural polymers (Radiation Processing of Polysaccharides International Atomic Energy Agency 2004 report). Sterilisation by E-beam has also been suggested as a means to avoid decreases in mechanical properties observed following gamma-irradiation of collagen and chondroitin 4-,6-sulphate biomaterials designed for the coverage of serious burns (Berthod et al. Clinical Materials 1994 15(4):259-65).

However, studies comparing the physicochemical and biodegradative properties of human amniotic membranes cross-linked via gamma radiation, E-beam radiation or glutaraldehyde chemical crosslinking showed a decrease in tensile strength and elongation at break of the amniotic membrane in both the gamma and E-beam irradiated membranes. This decrease in tensile strength and elongation at break of the amniotic membrane are suggested to be caused by scission of collagen chains through irradiation (Valentino et al. Archives of Otolaryngology—Head and Neck Surgery 2000 126(2):215-9).

E-Beam Sterilisation

E-beam sterilisation is rapidly growing in the medical industry as it offers many advantages over other sterilisation methods. As compared to EO sterilisation, E-beam radiation does not result in residues and the process takes minutes as opposed to days or even weeks. A conveyor or cart system moves the product being sterilised under the E-beam at a predetermined speed to obtain the desired electron dosage. The medical industry standard dosage is 25 kGy, but lower doses, for example at least 15 kGy and/or for example no more than 40 kGy, can be used dependent on bioburden levels in the product. Product can be sterilised in the finished packaged form and there is no need for further processing steps after radiation, unlike for EO sterilisation. There is also no holding period for the dissipation of residues after radiation unlike with EO.

Suitable E-Beam sterilisation conditions for use in the sterilisation step of the process of the present invention comprise an electron dosage of, for example, from 15 to 40 kGy, optionally at least 25 kGy, but lower doses can be used dependent on the desired bioburden levels in the product.

Drugs

The types of drugs suitable for use in or on the drug delivery implant of the first and second aspects of the present invention include compounds, optionally, but not essentially water-soluble compounds, such as the salts of amino amide anaesthetics, including lidocaine, bupivacaine, ropivacaine and mepivacaine and analgesics, in particular the soluble salts of narcotic analgesics including morphine, codeine, hydrocodone, hydromorphone and oxycodone. Other compounds that may be suitable for delivery by this system include certain anti-inflammatory drugs like NSAIDs (naproxen sodium, diclofenac sodium). Optionally, the drugs suitable for use in the drug delivery implant of the first and second aspects of the present invention include water-soluble compounds such as the salts of amino amide anaesthetics, including lidocaine, bupivacaine, ropivacaine and mepivacaine. Further optionally, the drug suitable for use in the drug delivery implant of the first and second aspects of the present invention is bupivacaine.

The types of drugs suitable for use in or on the drug delivery implant of the first and second aspects of the present invention include
Local Anaesthetics such as, but not limited to, lidocaine, prilocaine, bupivicaine and its single enantiomer levobupivacaine, ropivacaine, mepivacaine, dibucaine, as well as the pharmaceutically acceptable salts of any of these local anaesthetics, including the hydrochloride salts of all the above.
NSAID Analgesics such as, but not limited to, diclofenac sodium and potassium salts, ketoprofen and its active enantiomer dexketoprofen, naproxen and its sodium salt, ibuprofen and its sodium salt and its active entantiomer dexibuprofen, meloxicam, piroxicam, indomethacin, acetylsalicylic acid, as well as the pharmaceutically acceptable salts of any of these NSAID analgesics
Opioid and related analgesics such as, but not limited to, morphine and it salts including sulphate and hydrochloride, diamorphine and its hydrochloride salt, desomorphine, codeine and its salts including phosphate, sulphate and hydrochloride, hydrocodone and its bitartrate salt, hydromorphone and its hydrochloride salt, oxycodone, oxymorphone; fentanyl and its related analogues alfentanil, sufentanil, remifentanil, carfentanil and lofentanil; buprenorphine and its hydrochloride salt, tramadol and its hydrochloride and tartrate salts and tapentadol.
Chemotherapeutic agents, such as, but not limited to 5-Fluorouracil.
Anti-microbials

Specific embodiments of the invention will now be demonstrated by reference to the following general methods of manufacture and examples. It should be understood that these examples are disclosed solely by way of illustrating the invention and should not be taken in any way to limit the scope of the present invention.

### EXAMPLE 1

### Determination of Viscosity of a Collagen Dispersion

I. Materials
collagen implants, for example, sponges, or portion thereof, sampled to contain at least 140 mg collagen. The collagen dispersions do not contain added salt from, for example, a drug. More specifically, the collagen sponges used for the determination of the viscosity of the collagen dispersions do not contain added salt from, for example, a drug.
2 mmol HCl solution
35 ml centrifuge tubes

II. Equipment

1. Sample Preparation
High shear homogeniser (Ultra-turrax UT25 (IKA), 18 mm wide head)
Calibrated pH-meter (Cyber Scan PH310)
Calibrated Digital Thermometer (ReiTech RT200-02)
Calibrated Timer
Ultrasonic Bath (Bandelin Sonorex Super 10P, volume: 2 litres)

2. Viscosity Measurement
Brookfield DVIII+ Rheometer
Brookfield TC-501 circulating water bath with programmable controller
Spindle ULA, with ULA-31Y sample chamber (volume ˜70 ml empty, ˜20 ml with spindle inserted)
Brookfield Rheocalc32 Software, V3.1-1

III. Methodology

Sample Preparation

Using a scissors, cut the amount of implant, for example, sponge(s) containing 140 mg of collagen into small pieces (each measuring about 1×1 cm) into a 35 ml centrifuge tube. Add 25 ml of 2 mmol HCl to the centrifuge tube. Homogenise using the IKA UT25 Ultra-Turrax/18 mm shaft for 2-3 minutes until the dispersion is visually homogeneous, and the temperature of the dispersion has reached at least 38.5° C. and not more than 42° C. The time taken to achieve this is in the range 2 mins 10 secs and 3 mins 20 secs. Measure the pH and, if required, adjust down to not more than 3.5 using 1M HCl. If the pH is less than 3.5, then adjustment is not required. Degas the dispersion until no bubbles are visible (less than 2 minutes) using the sonic bath at maximum strength (˜480W) and room temperature.

Viscosity Measurement

Carefully pour 16 ml of the degassed dispersion into the ULA chamber and check the sample for bubbles. Remove bubbles using a plastic pipette, if necessary. Carefully introduce the ULA spindle into the ULA chamber. Place the ULA chamber into the temperature-controlled double-envelope and attach the spindle to the coupling nut. Input the settings for the viscosity measurement into the software:
Rotation speed: 20 rpm (shear rate=24 s⁻¹)
Temperature 30.0+/−0.5° C.
Equilibration time: 15 minutes
Number of data points/sample: 6

Data Reduction

Determine the viscosity for each sample until three consecutive values have a % RSD <5.0; the sample viscosity is defined as the mean value of these three data points. Using new implants or sponges, repeat the method until at least 10 (maximum 12) sample viscosity measurements are obtained. Calculate the average, standard deviation and % RSD of all sample replicates.

Using the above-mentioned methodology, the viscosity of unsterilised collagen sponges was determined and the data are set out in the table below:

Non-Sterile:

| | NS1 | NS2 | NS3 | NS4 | NS5 | NS6 | NS7 | NS8 | NS9 | NS10 | NS11 | NS12 |
|---|---|---|---|---|---|---|---|---|---|---|---|---|
| Viscosity | 161 | 164 | 151 | 141 | 145 | 149 | 173 | 149 | 146 | 150 | 163 | 141 |
| (mPas) | 169 | 160 | 155 | 142 | 152 | 161 | 178 | 142 | 154 | 142 | 163 | 140 |
| | 156 | 160 | 155 | 142 | 144 | 154 | 177 | 149 | 143 | 149 | 158 | 137 |
| Average | 162 | 161 | 153 | 142 | 147 | 155 | 176 | 147 | 148 | 147 | 161 | 139 |
| St Dev | 6 | 2 | 2 | 1 | 4 | 6 | 3 | 4 | 6 | 4 | 3 | 2 |
| CV (%) | 3.9 | 1.1 | 1.2 | 0.5 | 2.8 | 3.8 | 1.6 | 2.8 | 3.7 | 2.8 | 1.9 | 1.3 |

| | | | |
|---|---|---|---|
| | Average | St dev | % CV |
| n = 12 | 153 | 10 | 6.8 |

It will be observed that unsterilised sponges show an average viscosity of 153 milliPascals (mPas).

Using the above-mentioned methodology, the viscosity of collagen sponges sterilised by E-beam using Method C above by a radiation dose of at least 25 kGy was determined and the data are set out in the table below:

E-Beam:

| | EB1 | EB2 | EB3 | EB4 | EB5 | EB6 | EB7 | EB8 | EB9 | EB10 |
|---|---|---|---|---|---|---|---|---|---|---|
| Viscosity | 117 | 115 | 111 | 117 | 114 | 111 | 96 | 117 | 118 | 102 |
| (mPas) | 111 | 125 | 106 | 112 | 108 | 112 | 102 | 123 | 110 | 95 |
| | 119 | 115 | 112 | 120 | 113 | 113 | 100 | 114 | 121 | 99 |
| Average | 116 | 118 | 110 | 116 | 112 | 112 | 99 | 118 | 117 | 98 |
| St dev | 4 | 6 | 4 | 4 | 3 | 1 | 3 | 5 | 6 | 4 |
| CV (%) | 3.8 | 4.7 | 3.2 | 3.8 | 2.9 | 0.9 | 2.9 | 3.9 | 4.7 | 3.6 |

| | | | |
|---|---|---|---|
| | Average | St dev | % CV |
| n = 10 | 112 | 7 | 6.5 |

It will be observed that collagen sponges sterilised by E-beam show an average viscosity of 112 mPas.

Using the above-mentioned methodology, the viscosity of collagen sponges sterilised by EO under Method A above (specifically, evacuation down to −0.8 bar, following which the chamber is charged with sterilising gas (EO/CO₂ mixture, containing 15% EO w/w) up to between +4 bar (range 3.6-4.1 bar) for 6 hours (+/−10 minutes) at 32°-40° C. desorption is effected by alternating +0.6 bar overpressure and −0.8 bar underpressure for less than 12 hours) was determined and the data are set out in the table below:

EO Sterilised:

| | EO1 | EO2 | EO3 | EO4 | EO5 | EO6 | EO7 | EO8 | EO9 | EO10 | EO11 | EO12 |
|---|---|---|---|---|---|---|---|---|---|---|---|---|
| Viscosity | 188 | 189 | 156 | 189 | 205 | 169 | 175 | 176 | 179 | 192 | 187 | 152 |
| (mPas) | 193 | 196 | 161 | 194 | 191 | 162 | 169 | 190 | 173 | 206 | 170 | 157 |
| | 180 | 178 | 157 | 195 | 203 | 173 | 173 | 181 | 184 | 193 | 179 | 157 |
| Average | 187 | 188 | 158 | 193 | 199 | 168 | 172 | 182 | 179 | 197 | 179 | 156 |
| St dev | 6 | 9 | 2 | 3 | 7 | 6 | 3 | 7 | 5 | 8 | 9 | 3 |
| CV (%) | 3.4 | 4.7 | 1.5 | 1.6 | 3.7 | 3.3 | 1.8 | 4.0 | 2.9 | 4.0 | 4.7 | 1.8 |

| | | | |
|---|---|---|---|
| | Average | St dev | % CV |
| n = 12 | 180 | 14 | 7.9 |

It will be observed that collagen sponges sterilised by EO show an average viscosity of 180 mPas.

Using the above-mentioned methodology, the viscosity of collagen sponges sterilised by gamma irradiation at a radiation dose of at least 25 kGy was determined and the data are set out in the table below:

Gamma Sterilised:

| | G1 | G2 | G3 | G4 | G5 | G6 | G7 | G8 | G9 | G10 | G11 | G12 |
|---|---|---|---|---|---|---|---|---|---|---|---|---|
| Viscosity | 105 | 90 | 85 | 89 | 76 | 77 | 76 | 73 | 94 | 100 | 89 | 81 |
| (mPas) | 110 | 92 | 84 | 87 | 84 | 79 | 76 | 76 | 101 | 100 | 84 | 89 |
| | 102 | 94 | 90 | 87 | 84 | 86 | 81 | 71 | 102 | 97 | 86 | 82 |
| Average | 105 | 92 | 86 | 87 | 82 | 81 | 78 | 73 | 99 | 99 | 87 | 85 |
| St dev | 4 | 2 | 3 | 1 | 4 | 3 | 3 | 2 | 4 | 2 | 2 | 4 |
| CV (%) | 4.0 | 1.9 | 3.9 | 1.2 | 4.4 | 4.1 | 4.2 | 3.3 | 4.4 | 2.0 | 2.6 | 4.4 |

| | | | |
|---|---|---|---|
| | Average | St dev | % CV |
| n = 12 | 88 | 10 | 10.9 |

It will be observed that collagen sponges sterilised by gamma irradiation show an average viscosity of 88 mPas.

Summary viscosity data are presented below:

### Sterilisation Mode

| | | | | Gamma |
|---|---|---|---|---|
| Sample # | Non Sterilised | E-beam | EO | irradiated |
| S1 | 162 | 116 | 187 | 105 |
| S2 | 161 | 118 | 188 | 92 |
| S3 | 153 | 110 | 158 | 86 |
| S4 | 142 | 116 | 193 | 87 |
| S5 | 147 | 112 | 199 | 82 |
| S6 | 155 | 112 | 168 | 81 |
| S7 | 176 | 99 | 172 | 78 |
| S8 | 147 | 118 | 182 | 73 |
| S9 | 148 | 117 | 179 | 99 |
| S10 | 147 | 98 | 197 | 99 |
| S11 | 161 | — | 179 | 87 |
| S12 | 139 | — | 156 | 85 |
| Average | 153 | 112 | 180 | 88 |
| St dev | 10 | 7 | 14 | 10 |
| % CV | 6.8 | 6.5 | 7.9 | 10.9 |

### EXAMPLE 2

### T-Test: Is There a Statistically Significant Difference in Viscosity Between Types of Sterilisation?

H0: There is no significant difference between the viscosity of sponges from one Group when statistically compared to another.

H1: There is a significant difference between the viscosity of sponges from one Group when statistically compared to another.

The table below shows a t-test comparing the viscosity of EO sterilised collagen sponges with the viscosity of non-sterilised (NS) collagen sponges:

T-Test: Two-Sample Assuming Equal Variances

| | EO | NS |
|---|---|---|
| Mean | 180 | 153 |
| Variance | 204 | 109 |
| Observations | 12 | 12 |
| Pooled Variance | 156 | |
| Hypothesised Mean Difference | 0 | |
| df | 22 | |
| t_{Stat} | 5.21 | |
| P(T <= t) two-tail | 3E−05 | |
| t_{Stat} two-tail | 2.32 | |
| alpha | 0.03 | |

Because t_{Stat} is greater than t_{Critical} two-tail (Tc), H0 is rejected and, therefore, there is a statistically significant difference between the viscosity of NS sponges and the viscosity of EO sterilised sponges (T-test=2.32, df=22, p=3E-5).

The table below shows a t-test comparing the viscosity of non-sterilised (NS) collagen sponges with the viscosity of E-beam sterilised (EB) collagen sponges:

T-Test: Two-Sample Assuming Equal Variances

| | NS | EB |
|---|---|---|
| Mean | 153 | 112 |
| Variance | 109 | 53 |
| Observations | 12 | 10 |
| Pooled Variance | 84 | |
| Hypothesised Mean Difference | 0 | |
| df | 20 | |
| t_{Stat} | 10.57 | |
| P(T <= t) two-tail | 1E−09 | |
| t_{Stat} two-tail | 2.34 | |
| alpha | 0.03 | |

Because t_{Stat} is greater than t_{Critical} two-tail (Tc), H0 is rejected and, therefore, there is a statistically significant difference between the viscosity of NS sponges and the viscosity of EB sterilised sponges (T-test=2.34, df=20, p=1E-9).

The table below shows a t-test comparing the viscosity of E-beam sterilised (EB) collagen sponges with the viscosity of Gamma sterilised (G) collagen sponges:

T-Test: Two-Sample Assuming Equal Variances

| | EB | G |
|---|---|---|
| Mean | 112 | 88 |
| Variance | 53 | 92 |
| Observations | 10 | 12 |
| Pooled Variance | 75 | |
| Hypothesised Mean Difference | 0 | |
| df | 20 | |
| t_{Stat} | 6.42 | |
| P(T <= t) two-tail | 3E−06 | |
| t_{Stat} two-tail | 2.34 | |
| alpha | 0.03 | |

Because t_{Stat} is greater than t_{Critical} two-tail (Tc), H0 is rejected and, therefore, there is a statistically significant difference between the viscosity of EB sterilised sponges and the viscosity of G sterilised sponges. (T-test=2.34, df=20, p=3E-6).

In summary, the T-test can be used to show that there is a statistically significant difference between the viscosity of NS sponges and either the viscosity of EO sterilised sponges or the viscosity of EB sterilised sponges, as well as, between the viscosity of EB sterilised sponges and the viscosity of G sterilised sponges.

### EXAMPLE 3

### Comparison of EO Sterilisation and Gamma Radiation

Characterisation work was conducted to compare the effect of EO sterilisation and gamma radiation on the fibrillar collagen matrix using different analytical techniques. The results demonstrate that EO sterilisation of collagen leads to crosslinking in the collagen molecule whereas, in contrast, gamma radiation leads to chain scission within the collagen molecule.

Differential Scanning Calorimetry (DSC) Analysis

Differential Scanning Calorimetry (DSC) analysis was conducted on non-sterile, EO sterilised and gamma radiated placebo collagen sponges. The samples were compressed by hand and cut into small rectangular pieces. These pieces were then placed in aluminium pans and sealed. Two samples from the same sponge were tested for comparative purposes. The DSC used was a TA 2910 differential scanning calorimeter with sample sizes between 3.60 and 3.90 mg. All DSC tests were carried out under a 20 mL per minute flow of nitrogen to prevent oxidation. The resulting DSC scans (see FIG. 2) demonstrated that the thermal behaviour of the EO sterilised collagen samples is different to the gamma radiated samples, which in turn confirms that the thermodynamic properties of the collagen sponge matrix are dependent upon the method of sterilisation. In essence, the DSC profile for the EO product is similar to the non-sterile and, although both methods of sterilisation are likely to cause some breakdown of either the collagen molecule itself or crosslinks within the collagen structure, it is hypothesised that EO also creates more crosslinks at different points which may compensate for the breakdown. Whilst the unsterilised sponge behaves in a similar manner as the EO sterilised sponge with regard to drug release and collapse, the unsterilised sponge cannot be used as a surgical implant or wound care product. It will be appreciated that the drug delivery implants of the present invention need to be sterilised, in order to be used as surgical implants or as wound care products.

Hydration Studies

Collagen sponge samples, as detailed in Table 1, were weighed and placed into glass Petri dishes containing deionised water. These Petri dishes were then stored in an oven at 37° C. After a period of 24 hours, the Petri dishes were removed from the oven and the weight of each sample was recorded. This procedure was repeated every 24 hours for two weeks and each type of collagen sponge, Table 1, was tested in triplicate. The average weight increase at each timepoint was calculated.

TABLE 1
Samples used in the hydration studies
Collagen Sponges:

Non-sterilised collagen
EO sterilised collagen
Gamma sterilised collagen

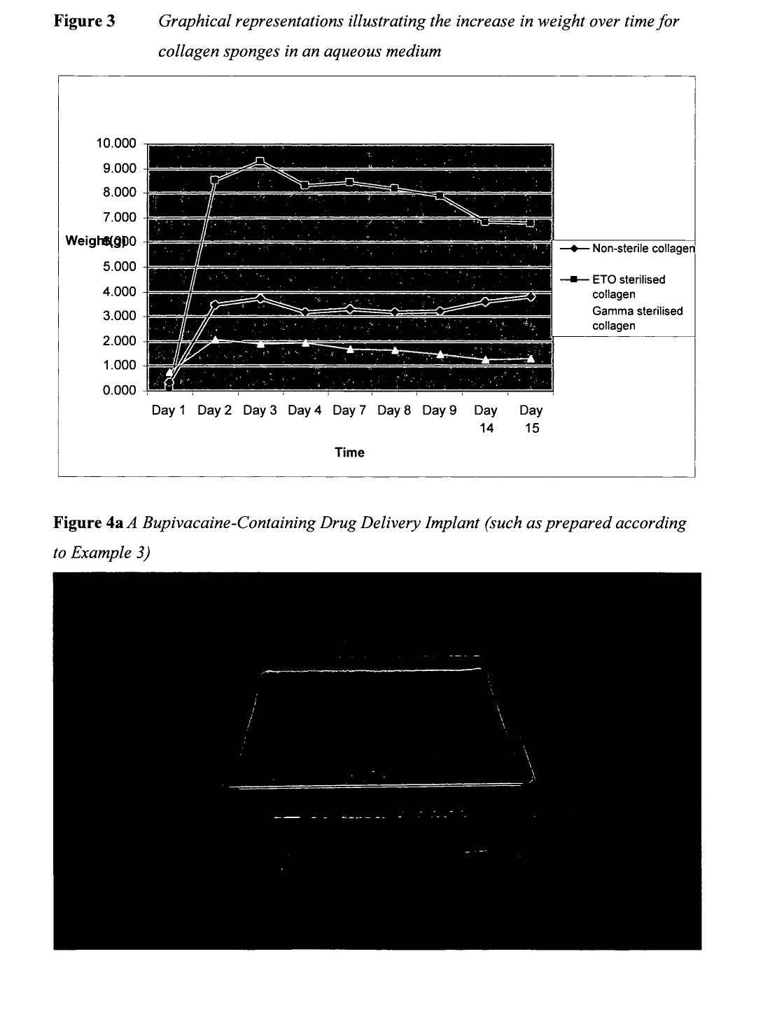

As expected, there was an increase in the overall weight of each sample as presented in FIG. 3. The breakdown of the fibrillar collagen structure by gamma sterilisation was evident in that this sample absorbed the least deionised water and, after a period of two weeks, the sample had lost much of its structural integrity. This suggests that gamma irradiation of the fibrillar collagen structure had initiated chain scission, resulting in a lower molecular weight material with fewer crosslinks. This corresponds with the findings of Noah, E. M. et al (2002). The largest weight increase occurred in the samples containing the EO sterilised collagen. This would suggest that the EO sterilised collagen sponges have an increased crosslinked density when compared to the gamma sterilised sponges.

Viscosity Studies

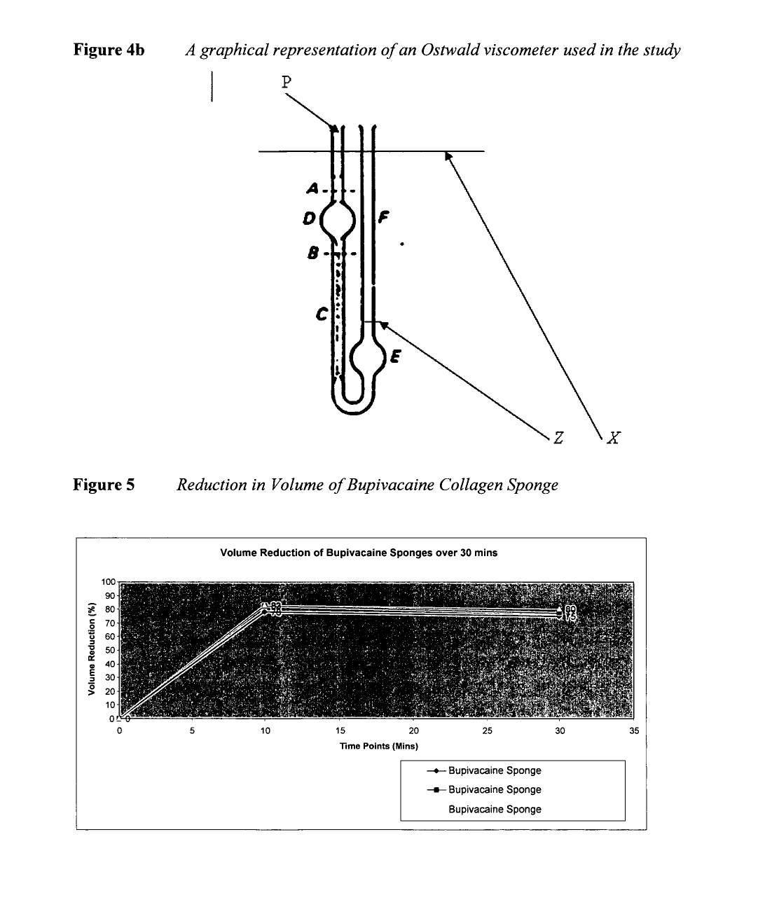

Viscosity studies were performed on each of the collagen sponge samples as detailed in Table 1 using a B type Ostwald viscometer at 37° C. The viscometer was rinsed several times with deionised water and allowed to dry in an oven at 80° C. The viscometer was then cooled to room temperature after which time it was filled between the marks, B and Z as depicted in FIG. 4b, with deionised water. The viscometer was then placed into a temperature controlled water bath at 37° C. and the solution was allowed to equilibrate to the required temperature. Using a pipette bulb, connected at P, the solution was sucked above the graduation mark ‘X’ after which the pipette bulb was released. When the meniscus passed “A”, a stopwatch was started until it reached point “B” after which time the stopwatch was stopped and the flow time was recorded. This procedure was repeated three times and the average value was used. This value is known mathematically as “to”.

The viscometer was then washed and dried in an oven at 80° C.

The collagen sponge samples were prepared as follows: 0.56 g of the sponge and 0.2 ml of acetic acid were placed in 99.2 ml of deionised water; the solution was then heated to approximately 37° C. The pH value was monitored, 4.5+/−0.2 and, when applicable, 0.1M sodium hydroxide solution was added to bring the solution to the required pH for homogenisation. The samples were then homogenised using a suitable blender. This procedure was repeated for each of the collagen sponges and the homogenised solutions were then analysed using the Ostwald viscometer as previously described. The average values obtained are known mathematically as “tsol”.

Using the values obtained, to and tsol, the relative and specific viscosity were calculated for each sponge, where

Relative viscosity ηᵣ=tsol/to (eq 1)

Thus the results obtained from this study are presented in Table 2.

TABLE 2
Viscosity results obtained for each of the collagen sponges
tested at 37° C.

| Samples | ηᵣ |
|---|---|
| EO sterilised collagen sponge | 4.47 |
| Gamma sterilised collagen sponge | 1.28 |
| Non-sterilised collagen sponge | 1.6 |

As illustrated in Table 2, the relative viscosity result for the gamma sterilised collagen sponge is lower than the result obtained for the non-sterilised collagen sponge. In addition, the relative viscosity results for the non-sterilised sponge is lower than the result obtained for the EO sterilised sponge. Whilst the DSC data of Example 1 suggest that EO sterilised and non-sterilised collagen sponges are comparable, the relative viscosity data would seem to suggest that the EO sterilised collagen sponge was more crosslinked than the other collagen sponges tested and this trend was also evident from the results of the hydration studies (FIG. 3).

Studies comparing the physicochemical and biodegradative properties of human amniotic membranes crosslinked via gamma radiation or by chemical means via glutaraldehyde showed a decrease in tensile strength and elongation at break of the amniotic membrane in both the gamma and E-beam irradiated membranes. This decrease in tensile strength and elongation at break of the amniotic membrane are suggested to be caused by scission of collagen chains through irradiation (Valentino et al. Archives of Otolaryngology—Head and Neck Surgery 2000 126(2):215-9).

### EXAMPLE 4

### Effect of Crosslinking by Glutaraldehyde on the Physical Properties of Fibrillar Collagen Matrices

Another means of creating crosslinks in fibrillar collagen matrices is to use chemical crosslinking agents, such as glutaraldehyde, carbodiimides and organic peroxides. To assess the effect of crosslinking on the physical properties of fibrillar collagen matrices, the chemical crosslinking agent, glutaraldehyde (GTA), was added to collagen dispersions at a 1% collagen concentration and brought to pH 4.5±0.2 at levels of 0 to 0.8% w/w of the collagen content (see Table 3—the glutaraldehyde concentration is expressed as a percentage of the collagen to be crosslinked).

After the GTA was added, the pH was brought to the standard pH 4.5±0.2 and drug delivery implants in the form of collagen sponges were manufactured by lyophilisation followed by gamma radiation at 32 kGy. The tensile strength of the resulting matrices was measured in wet form and showed an increase in tensile strength with increase in glutaraldehyde and likely correlating with increased of crosslinking (Table 3).

TABLE 3
Tensile Strength in Glutaraldehyde Crosslinked Collagen Sponges

| | GTA Conc % of collagen | | | | |
|---|---|---|---|---|---|
| | 0 | 0.1 | 0.2 | 0.4 | 0.8 |
| Wet Tensile Strength | 0.55 | 0.63 | 0.77 | 0.72 | 1.12 |
| (N) | | | | | |

It is postulated that changes in the density and porosity of the matrix through crosslinking (by EO sterilisation, dehydration or chemical means) leads to changes in drug release profiles.

### EXAMPLE 5

### Bupivacaine-Containing Drug Delivery Implant (Bupivacaine Collagen Sponge)

This drug delivery implant according to the first and second aspects of the invention is composed of a highly purified Type I collagen matrix containing the amide local anaesthetic bupivacaine hydrochloride (HCl). The system is terminally sterilised by ethylene oxide to yield a sterile matrix suitable for surgical implantation.

Method of Manufacture

Collagen Extraction and Purification

Collagen can be extracted from a number of sources including animal hides and animal tendons. The collagen used in the drug delivery implant of the first and second aspects of the invention is derived from animal tendon (equine or bovine) and more preferably from bovine tendon. The extraction process for collagen is according to standard practice and well known to those skilled in the art. During the manufacturing process, milled bovine tendons are treated with a number of reagents, most importantly, with 1N sodium hydroxide (NaOH) to remove microbiological contamination, including prions.

Further reduction of the particle size of the collagen-containing material is followed by treatment with pepsin at approximate pH of 2.5, which is used to degrade contaminating serum components, primarily bovine serum albumin and causes detachment of non-helical portions of the collagen molecule (telopeptides). During this process the collagen material is partially solubilised in the acidic medium. After filtration, precipitation of the collagen is accomplished by means of manipulation of the pH (from pH of about 2.5 to pH of about 7.5). The fibrillar collagen material is finally precipitated out of solution and then concentrated by means of centrifugation.

Compounding Process and Equipment

The collagen dispersion is manufactured in a stainless steel vessel. The aqueous based dispersion is prepared using pre-heated (35-42° C.) water and adjusted to pH 4.5±0.2. Without being bound by theory, it is thought that, when the bupivacaine is added to the collagen suspension at pH 4.5, the collagen sediments out and to reverse this, the pH is dropped to 3.9 to allow the collagen to become re-dispersed (fibres are more swollen and solubility increases at the lower pH). A pH of 3.9 is used for the equine collagen suspension to prevent precipitation but this is probably due to the higher level of cross-links that occur naturally over time (horses tend to be a lot older than the cattle used as tendon source).

High shear mixing is required to break up the collagen mass and expose the collagen fibres to the acidic medium. The homogeniser employed possesses a rotor-stator head that is designed to create high shear forces by pulling the material through the rotating homogeniser head and forcing it against the proximal stationary stator head. It is this design that facilitates the high shear forces required to separate the fibrous collagen mass at the beginning of suspension preparation.

The rotor/stator equipment used in the manufacturing process was selected based on existing in-house experience with this equipment. For example, an IKA Ultra-Turrax mixer may be used at a high speed for about 5-10 minutes, followed by low shear mixing after the addition of the drug for a minimum of 20 minutes.

Following completion of collagen suspension formation, the suspension is transferred to a closed heat jacketed stainless steel vessel for final compounding. The jacket temperature is maintained at 37° C. The bupivacaine HCl is first dissolved in a portion of water under manual stirring; this solution premix is then introduced into the heat jacketed SS vessel under low shear mixing to achieve homogeneity in the drug-loaded collagen suspension. The final drug/collagen suspension is adjusted to pH 3.9±0.3 prior to filling into blister trays or lyophilisation moulds and the collagen concentration is 0.56 g/100 ml. The pH of 3.9±0.3 was selected for the bupivacaine-collagen suspension to prevent the collagen sedimentation that would occur on addition of the drug. This pH supports the suspension of the collagen in the aqueous medium.

Filling/Lyophilisation Process and Equipment

The filling process is performed using a positive displacement pump. The pump is valve-less, has ceramic pistons and works on the principle of positive displacement.

Alternatively, a peristaltic pump could be used. The drug-collagen suspension can be filled into lyophilisation moulds or into blister trays. 12.5 grams of the drug-collagen suspension (containing the equivalent of 50 mg bupivacaine HCl) is filled into a mould or blister with inner dimensions of 5 cm×5 cm×1.3 cm. Upon completion of tray filling, the filled moulds/blisters are placed into the lyophiliser. A commercially available lyophiliser (e.g. Christ lyophiliser) was utilised for lyophilisation of the bupivacaine-containing drug delivery implant. The lyophiliser operating console permits process cycle programming and an automated program cycle was established for the product to yield the lyophilised sponge in FIG. 4a with approximate dimensions of 5 cm×5 cm, with a thickness in the range of about 3.5 to about 5.0 mm.

Packaging Process and Equipment

Following completion of the lyophilisation cycle, the trays are removed from the shelves. If lyophilisation occurred in moulds, the sponges are removed from the moulds and packed into blister trays. The packaging process proceeds in two steps, inner blister packaging and sealing and outer (EO-type) pouch packaging and sealing. Sponge product is placed into inner PETG blisters and the blisters are then sealed with a Tyvek lid using pneumatic blister packing/sealing equipment. When lyophilisation occurs in the blister tray, there is no requirement for removal from the blister, so packaging is initiated with sealing of the blister. The sealed blister is then inserted into an outer EO sterilisable pouch. One side of the outer pouch consists of a transparent polyester/LDPE foil laminate with a Tyvek strip seal while the other side an opaque polyester/LDPE laminate. Other outer pouch packaging can be used including aluminium oxide coated polyethylene materials or if E-beam radiation is used for sterilisation, an aluminium outer pouch can be used. The pouch is then sealed using continuous heat sealing equipment. This heat sealer facilitates the formation of a continuous seal at the open end of the pouch. The top part of the pouch includes two holes or a strip lined with Tyvek. These windows are specifically designed for the EO gas sterilisation process and are gas permeable only. The permeability of the windows facilitates permeation of EO gas during the terminal sterilisation process. The sealed product is transferred to the terminal EO sterilisation process stage. Following sterilisation and ventilation, the outer pouch is resealed below the gas permeable windows and this gas permeable (top) portion is then removed from the pouch. This results in a fully sealed outer pouch containing a terminally sterilised sponge product.

EO Sterilisation Process and Equipment

Ethylene Oxide (C₂H₄O) is a gas at operating temperature and sterilises via its action as a powerful alkylating agent. Under the correct conditions, cellular constituents of organisms such as nucleic acid complexes, functional proteins and enzymes will react with ethylene oxide, causing the addition of alkyl groups. As a result of the alkylation, cell reproduction is prevented and cell death ensues. Specific processing conditions and parameters must be met to achieve this effect within the target product; including, but not limited to, acceptable concentration of ethylene oxide in the chamber and a minimum water activity level within the organism.

The steriliser is a Model DMB 15009 VD (made by DMB Apparatebau GmbH of Germany), a two-door fully automated unit with a chamber capacity of 1500 l. A mixture of EO/CO₂ at a ratio of 15:85 is used as the sterilisation gas. After sterilisation, the ethylene oxide is disposed of via two Donaldson catalytic converters.

Pre Sterilisation Equilibration

Sponges are prepared for sterilisation in a holding area with controlled environmental conditions (22±2° C. and RH 25-65%) to allow the moisture level in the sponge to equilibrate. The desired moisture limit of Not Less Than 9% moisture (determined by Loss on Drying) must be achieved in the sponge prior to sterilisation.

Loading the Chamber

The pouched sponge product is positioned in rows within stainless steel mesh baskets and placed in the sterilisation chamber. This ensures that the sterilisation gas is uniformly distributed in the chamber and that all sponges are exposed to an equivalent gas concentration.

The sterilisation process is based on a 4-bar pressure cycle, which is maintained for a sterilisation period of 6 hours, as described below. The steriliser is completely automatic, which means that the chamber can only be opened after completing the full sterilisation cycle.

Cycle Parameters

The sterilisation cycle can be subdivided into the following phases:

High-Pressure Test:

System check in the high-pressure range; the cycle will be aborted if any leaks occur.

Low-Pressure Test:

The chamber is charged with compressed air up to the sterilisation pressure of 4 bar and maintained at this pressure for 5 minutes. The cycle will be aborted if leaks from the chamber are detected. If the test is successful, the compressed air is released and the chamber is evacuated to −0.8 bar.

Gas Feeding:

The system switches immediately to gas feed once the necessary vacuum has been achieved. The liquid EO/CO₂ mixture is evaporated and conditioned by the two gasifiers and the introduction of the gas into the chamber takes approximately 15 minutes for this 4 bar cycle.

The chamber heating system is activated during the preliminary program. All parameters for starting of sterilisation are fulfilled by the end of the gas feed phase.

Sterilisation:

Sterilisation time: 6 hours (half-time cycle: 3 hours)

Chamber pressure: 4 bar (limits: 3.6 to 4.1 bar)

Chamber temperature: 30° C. to 40° C.

Ethylene oxide concentration: >1300 mg ETO/l

Gas Discharge:

Discharge of the sterilising gas from the chamber is accomplished under volumetric control via two Donaldson catalytic converters.

Desorption:

Desorption of the gas occurs in the chamber. The temperature for this phase is the same as for sterilisation (30° C. to 40° C.). The chamber is alternately evacuated (to −0.8 bar) and flushed with compressed air. A pressure of around 0.6 bar is achieved at this stage. This cycle is repeated every 30 minutes. Desorption takes not less than 12 hours; the chamber remains locked (interlock) throughout this period.

Longer Term Aeration

This phase of the process serves to further scavenge low-level residual ethylene oxide from the product and packaging. The product is held at room temperature until the limits for ethylene oxide derivative residues have been reached, for a minimum of 3-4 weeks.

### EXAMPLE 6

### Collagen Sponge Collapse

In order to assess this characteristic of the present invention, the following experimental work was conducted using bupivacaine-containing drug delivery implants of Example 3 and also the collagen sponge drug delivery matrix without drug.

Shrinking of Collagen Sponges in PBS (Phosphate Buffer Saline solution) pH 6.8, at 37° C.

The experiment was performed in triplicate using three samples of Bupivacaine Collagen Sponges (5×5 cm) from different manufacturing lots, where each sample sponge comprised of 70 mg of collagen and 50 mg of bupivacaine HCl. It was also performed with a single manufactured lot of the drug delivery matrix without drug comprised of 70 mg of collagen. 50 ml of PBS (prepared according to Ph. Eur. 5^{th} Edition Volume 1, 01/2005 Reagents/Buffers Solutions 4.1.3) was placed in a 400 ml beaker and equilibrated in a water bath maintained at a temperature of 37° C. The general appearance and dimensions (length, width and thickness) of each dry sponge sample was recorded before it was placed in the PBS in the 400 ml beaker.

At time points of 10 minutes and 30 minutes, the appearance and dimensions were observed, measured and recorded. The length and width of the sponge were measured by placing the sponge (while still in the beaker) over a scaled 50×50 mm² template. The thickness was recorded by removing the sponge from the beaker using tweezers and measured using manual callipers (mm).

Results:

TABLE 4
Sponge Collapse Measurements
Volume Reduction of Bupivacaine HCl Collagen Sponge
(50 mg drug in 70 mg sponge) (5 × 5 cm)
upon buffer uptake at 37° C.

| | Sample No. | | |
|---|---|---|---|
| | 1 | 2 | 3 |
| | Volume reduction | | |
| Time min | in % | | |
| 0 | 0 | 0 | 0 |
| 10 | 78 | 82 | 83 |
| 30 | 75 | 77 | 80 |

TABLE 5
Sponge Collapse Measurements
Volume Reduction of 70 mg Collagen Sponge (5 × 5 cm)
upon buffer uptake at 37° C.

| Time min | Volume reduction in % |
|---|---|
| 0 | 0 |
| 10 | 78 |
| 30 | 70 |

The results indicate a reduction in volume of the drug delivery implant by at least 70% on absorption of fluid after 10 minutes. This reduction in volume facilitates the use of the implant as a drug delivery system for the local administration of pharmacological agents. The results are presented graphically in FIG. 5

### EXAMPLE 7

### Preclinical Data. Pharmacokinetics. Abdominal Implantation

Both preclinical (beagle dog model) and human clinical studies have been conducted on the bupivacaine-containing drug delivery implant of the present invention and the blood plasma concentrations consistently demonstrate a double peak phenomenon.

Preclinical Data

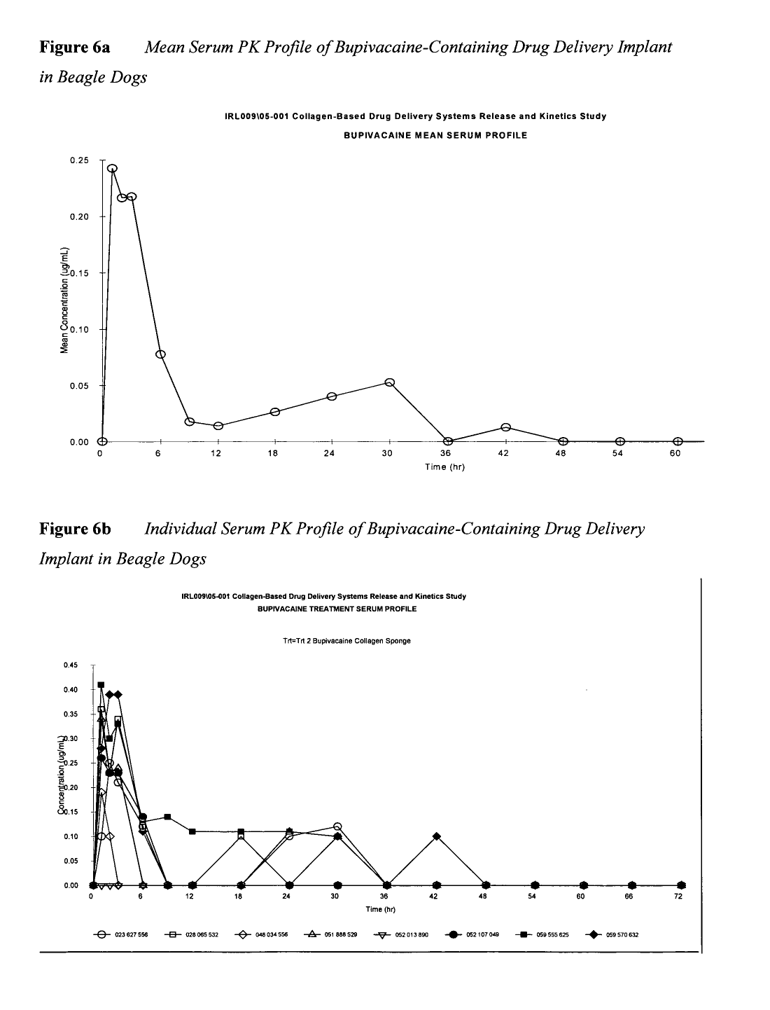

In a preclinical study, an EO sterilised bupivacaine-containing drug delivery implant (50 mg bupivacaine HCl in a 5×5 cm collagen sponge matrix) was implanted into the abdomen of 8 beagle dogs (4 male and 4 female). Blood sampling took place prior to test item administration and at the following time-points (±10 minutes): 1, 2, 3, 6, 9, 12, 18, 24, 30, 36, 42, 48, 54, 60, 66, 72 hours. The mean PK profile indicates two peaks in serum concentration of bupivacaine occurring at 2 hours and 30 hours post-implantation, as illustrated in FIG. 6a. The individual serum profiles in FIG. 6b also reveal a double peak, proving that the double peak exhibited by the mean profile is a function of drug release from the implant and not an artefact of having either a fast or slow time to maximum concentration in the different animals.

### EXAMPLE 8

### Preclinical Data. Pharmacokinetics. Comparison of Sterilisation Method

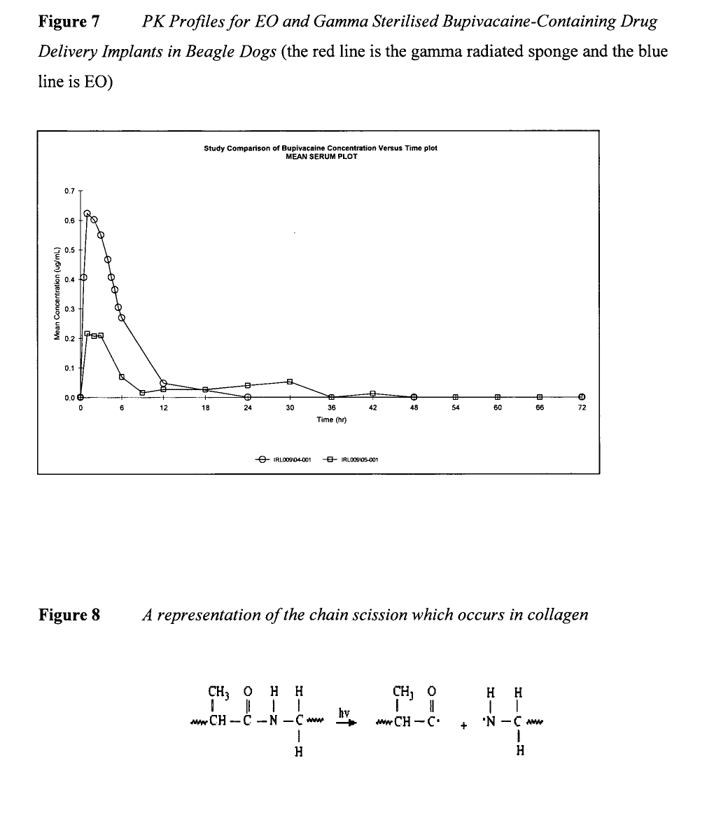

The influence of the sterilisation method was evaluated by cross comparing PK profiles of an EO treated bupivacaine-containing implant with a gamma radiated bupivacaine-containing implant (50 mg bupivacaine HCl in a 5×5 cm 70 mg collagen sponge matrix) from two different preclinical beagle dog studies. In the case of the EO sterilised implant, implantation was made in the abdomen whereas the gamma sterilised implant was implanted in the hind leg. The site of implantation may play a role because the site itself will influence level of fluid and access to circulation (see Example 7 below where the site of implantation is solely the abdomen). PK sampling was conducted in a similar manner between the two studies. FIG. 7 shows the PK profiles for both drug delivery implants; EO sterilised in blue and gamma sterilised in red.

A distinct difference in PK profiles is observed between the two implants. The gamma sterilised implant shows a typical single peak profile whereas the EO sterilised implant exhibits a double PK peak. The differences in the PK profiles can be explained by the influence of the sterilisation method on the fibrillar collagen matrix and particularly on the extent of crosslinking in the collapsed collagen sponge which modifies the drug release kinetics.

Studies have shown that gamma irradiation brings about chain scission of the collagen molecule causing polymer fragmentation, FIG. 8. Typically, some manufacturers compensate for this increased fragmentation by deliberately crosslinking the protein. It is thought that the majority of gamma-irradiation damage is induced by free radicals resulting from the radiolysis of water molecules. However, gamma irradiation leads to significant collagen sponge degradation (whether crosslinked or not) and to a decrease in denaturation temperature and tensile strength (Salehpur et al 1995, Liu et al 1989, Noah et al 2002).

It is postulated that this more rapid degradation of the gamma sterilised sponge results in a faster release of drug from the matrix in vivo, which subsequently influences the systemic (PK) profile.

EO sterilisation is known to induce crosslinking in the collagen molecule and amino acid analysis indicates intensive reaction of EO with the amino-groups presented by collagen in the form of lysine and hydroxylysine residues (Friess 1998).

It is postulated that the first PK peak observed in the preclinical assessment can be explained by the passive dissolution of the drug from the surface of the implant. Once the implant absorbs fluid and the structure collapses, a hydrogel-type material is formed, which is strengthened by the crosslinking induced by the EO sterilisation process. Drug release occurs by diffusion through the hydrogel layer and is subsequently delayed resulting in the second peak observed in the PK profile. Conversely, in the case of gamma sterilisation, the reduced level of crosslinking produces a weaker (lower viscosity) hydrogel meaning the drug is released more rapidly and only a single peak is observed. The double PK peak observed in the preclinical assessment also appears in humans—see Example 8.

### EXAMPLE 9

### Preclinical Data. Comparison of EO and E-beam Sterilisation

A study was conducted to compare the local and systemic pharmacokinetics of three bupivacaine-containing drug delivery implants, two of which were EO sterilised but had been stored for different time periods and the third was sterilised by E-beam radiation.

There were three groups in the study and each group contained 8 animals (4 males and 4 females). Animals were assigned to either Group 1 (EO-sterilised, bupivacaine-containing drug delivery implant after 2 years storage at ambient conditions), Group 2 (EO-sterilised bupivacaine-containing drug delivery implant recently manufactured) or Group 3 (E-beam sterilised bupivacaine-containing drug delivery implant). The implant was surgically inserted into the left lateral aspect of the abdomen. Blood samples (prior to test item administration and up to 72 hours post test item administration) were collected and assayed for bupivacaine concentration to determine the systemic PK profile of the three different implants.

The systemic PK profiles of the three implants showed a similar pattern in that a double peak in serum bupivacaine levels was observed in each case as illustrated by FIG. 12. This suggests that, unlike gamma sterilisation, E-beam sterilisation appears to lead to some level of crosslinking in the collagen matrix, thus resulting in a double PK peak due to the drug release kinetics from the matrix. One of the primary uses for E-beam radiation in industry is polymer crosslinking to enhance tensile strength and durability. This crosslinking effect is also observed within the collagen matrix of the current invention, as evidenced by its relative viscosity and the serum PK profile. E-beam sterilisation has also been suggested as a means to avoid decreases in mechanical properties observed following gamma irradiation of collagen and chondroitin-4,6-sulphate biomaterials designed for the coverage of serious burns (Berthod et al. Clinical Materials 1994 15(4):259-65).

### EXAMPLE 10

### Clinical Data

A Phase I single-dose open-label prospective study to investigate the pharmacokinetic (PK) profile, safety and tolerability of a bupivacaine-containing drug delivery implant of Example 3 in 12 patients following hysterectomy surgery was undertaken in the United Kingdom.

Three EO sterilised bupivacaine-containing drug delivery implants, each containing 50 mg bupivacaine HCl, were implanted at different levels in the surgical wound. One sponge was placed in the surgical vault, one at the peritoneum and the third under the incision in the dermis. This mode of administration allows for pain relief at the different sites where pain is experienced after this type of surgery. The primary objectives of this study were to determine the pharmacokinetic profile, safety and tolerability of a bupivacaine-containing drug delivery implant. Secondary objectives were the measurement of pain relief afforded by the implant by assessing morphine sparing (reduction in level of morphine consumed post-operatively over the first 24 hours) and Visual Analogue Scoring (VAS) of pain intensity. After surgery, each patient received PCA (patient-controlled analgesia) morphine for first 24 hours plus standard of care analgesia and the cumulative amount of morphine self-administered by the patient over that period was recorded. Pain intensity measured by VAS (at rest) on a scale of 0 to 100mm (0 is no pain and 100 is intolerable pain) was assessed at frequent intervals through to hospital discharge (at least 96 hours post-op).

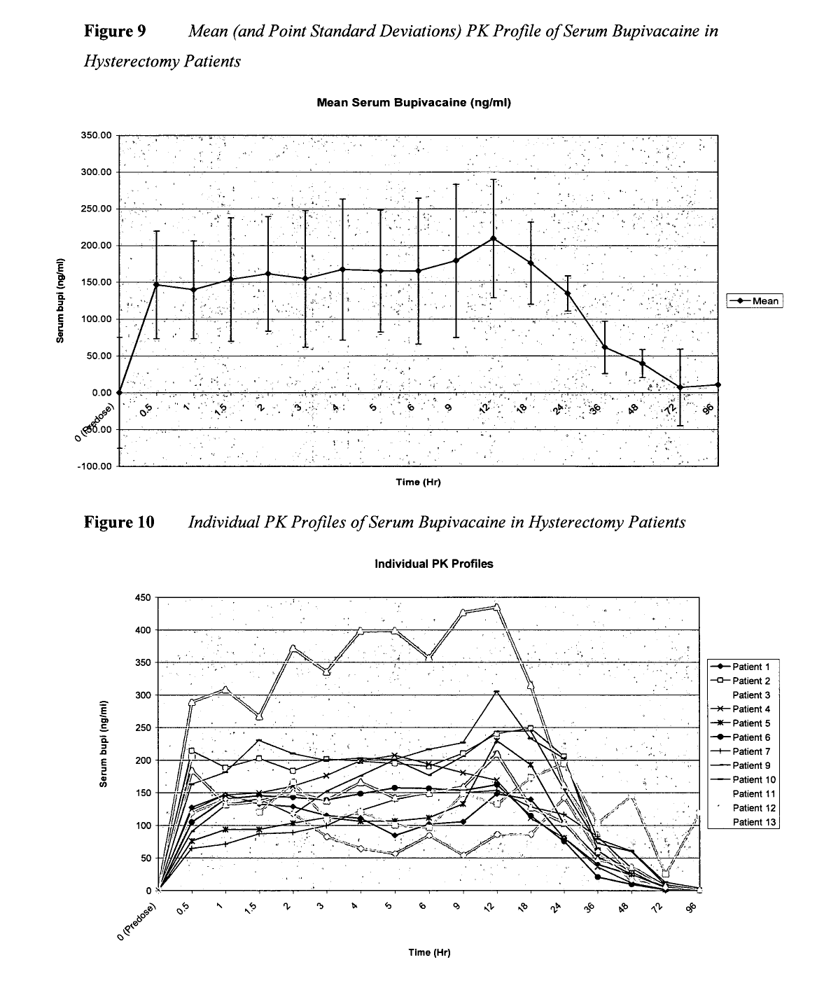

The mean and individual PK profiles of FIGS. 9 and 10, respectively, were determined by taking blood samples at baseline (prior to implantation of the implant) and at 30 mins, 1, 1.5, 2, 3, 4, 5, 6, 9, 12, 18, 24, 36, 48, 72 and 96 hours post-implantation of the implant. Plasma was separated from the blood and assayed for bupivacaine using a validated assay method.

The PK data from the hysterectomy trial indicates that most patients show a double peak of bupivacaine concentration in the systemic circulation. The first peak generally occurs within the first 1.5 hours and the second peak occurs between 12 and 18 hours, as illustrated in FIGS. 9 and 10. Whilst the half-life of bupivacaine used in regional anaesthesia is reported at about 3 hours (range of 1.5 to 5.5 hours), the drug delivery implants of the present invention demonstrate sustained local delivery of bupivacaine, with the second peak occurring well after the half-life of the drug.

A general outcome from the study was that all patients were, most unusually, mobilised within 24 hours post-op, which is an important step to rehabilitation after surgery.

Furthermore, low mean morphine consumption, (as compared to literature data) was reported and low VAS pain scores were recorded through to hospital discharge. The results indicate a prolonged duration of action of bupivacaine in the collagen drug delivery system (see Table 6 below).

TABLE 6
Summary of PCA Morphine Usage and Pain Scores of First 12 Subjects Enrolled in Ongoing Clinical Study

| Patient no | | | 001 | 002 | 003 | 004 | 005 | 006 | 007 | 008 | 009 | 010 | 011 | 012 |
|---|---|---|---|---|---|---|---|---|---|---|---|---|---|---|
| Age | | | 35 | 45 | 45 | 48 | 44 | 45 | 44 | 41 | 42.00 | 34 | 40 | 40 |
| Date of enrolment | | | 26 Jan. | 02 Feb. | 08 Feb. | 08 Feb. | 16 Feb. | 23 Mar. | 23 Mar. | 13 Apr. | 39206.00 | 11 May | 25 May | 13 Jul. |
| (date of surgery) | | | 2007 | 2007 | 2007 | 2007 | 2007 | 2007 | 2007 | 2007 | | 2007 | 2007 | 2007 |
| Total Amount of | | | 28 | 7 | 0 | 1 | 10 | 6 | 2 | 57* | 15.00 | 2 | 74** | 0 |
| Morphine consumed | | | | | | | | | | | | | | |
| (mgs) | | | | | | | | | | | | | | |
| VAS Pain | 1 | h | 68 | 45 | 2 | 19 | 36 | 34 | 22 | 48 | 5.32 | 0.00 | 100.00 | 17.02 |
| scores | 1.5 | h | 49 | 34 | 7 | 19 | 31 | 32 | 24 | 55 | 0.00 | 47.87 | 72.34 | 18.09 |
| (mm) | 2 | h | 43 | 56 | 13 | 30 | 27 | 25 | 41 | 34 | 29.79 | 28.72 | 80.85 | 17.02 |
| | 3 | h | 43 | 65 | 13 | 24 | 0 | 14 | 41 | 21 | 50.00 | 29.79 | 84.04 | 9.57 |
| | 6 | h | 16 | 5 | 0 | 10 | 0 | 17 | 5 | 12 | 14.89 | 10.64 | 34.04 | 2.13 |
| | 9 | h | 4 | 2 | 0 | 12 | 4 | 9 | 3 | 2 | 10.64 | 12.77 | 34.04 | 1.06 |
| | 12 | h | 4 | 0 | 6 | 6 | 1 | 13 | 20 | 2 | 3.19 | 13.83 | 23.40 | 5.32 |
| | 18 | h | ND | 0 | 4 | 6 | 8 | 65 | 24 | 8 | 1.06 | 7.45 | 21.28 | 14.89 |
| | 24 | h | 14 | 3 | 2 | 4 | 0 | 25 | 1 | 0 | 0.00 | 3.19 | 26.60 | 3.19 |
| | 36 | h | 24 | 2 | 3 | 0 | 21 | 11 | 0 | 0 | 0.00 | 3.19 | 4.26 | 2.13 |
| | 48 | h | 2 | 0 | 0 | 0 | 7 | 3 | 0 | 0 | 0.00 | 0.00 | 22.34 | 1.06 |
| | 72 | h | 1 | 9 | 0 | 0 | 2 | 11 | 6 | 0 | 0.00 | 0.00 | 2.13 | 0.00 |
| | 96 | h | 0 | 6 | 0 | 0 | 1 | 7 | 11 | 0 | 0.00 | 0.00 | 0.00 | 0.00 |

| | | | | | | | | | | | | |
|---|---|---|---|---|---|---|---|---|---|---|---|---|
| Day of discharge | 5 | 4 | 5 | 5 | 6 | UNK | 4 | 6 | 6 | 5 | 5 | 4 |

*Patient suffered from an underlying lower back pain problem
**Patient received non-standard anaesthesia/analgesia

### CONCLUSION

The drug delivery implant of the present invention, based on a fibrillar collagen matrix, is ideal as an implantable matrix for the local delivery of drugs. The fibrillar collagen sponge matrix structure collapses once fluid is absorbed, thus reducing the matrix volume and avoiding any issues of pressure being applied to surrounding tissues and nerves. The collapsing nature of the fibrillar collagen sponge matrix is highly desirable and is unlike commercially available collagen based haemostats, which swell on absorbing fluid and lead to serious complications, which have resulted in restriction of use issued by the FDA. As a result of the structure collapse in vivo, the drug delivered by the drug delivery implant of the present invention demonstrates a double peak in the systemic drug level as illustrated by the PK profile. This double peak phenomenon may provide for an extended duration of action, which is particularly desirable in the case of local drug delivery for pain relief.

### REFERENCES

K. Gelse et al, Collagens—structure, function and biosynthesis. Adv. Drug Delivery Rev., 55 (12): 1531-46 (2003).

M. E. Nimmi, R. D. Harkness, Molecular structure and functions of collagen, in: M. E. Nimmi (Ed.), Collagen vol. I, CRC Press, Boca Raton, Chapter 1: 1-77 (1988).

K. Anselme et al, Tissue reaction to subcutaneous implantation of a collagen sponge. J. Biomed. Mater. Res., 24 (6): 689-703 (1990).

A. E. Postlethwaite et al, Chemohaemotactic attraction of human fibroblasts to collagen, Proc. Natl. Acad. Sci., 75(2): 871-875 (1978).

K. E. Burke & G. Naughton, Demonstration by immunofluorescence that host collagen replaces dermal microimplants of bovine collagen. J. Dermatol. Surg. Oncol., 9: 889-895 (1983).

D. M. Cooper, The physiology of wound repair, in: D. Krasner (Ed.), Chronic Wound Care, Health Management Publications, King of Prussia, Pa., Chapter 7.

L. Leipziger et al, Dermal wound repair: the role of collagen matrix implants and synthetic polymer dressings. J. Am. Acad. Dermatol., 12 (2Pt2): 409-419 (1985).

K Patterson & R. E. Numerof, Drugs and devices working in combination, Scrip Magazine, PJB Publications, December 2003.

Y. Tomizawa, MD Clinical benefits and risk analysis of topical haemostats: a review. J Artif Organs 8:137-142 (2005).

S. Iwabuchi, K. Koike, T. Okabe, S. Tago, T. Murakami Iatrogenic paraplegia caused by Surgicel used for haemostasis during a thoracotomy: report of a case. Surg Today 27:969-970 (1997).

A. R. Brodbelt, J. B. Miles, P. M. Foy, J. C. Broome Intraspinal oxidized cellulose (Surgicel) causing delayed paraplegia after thoracotomy—a report of three cases. Ann R Coll Surg Engl 84:97-99 (2002).

R. Z. Lovstad, P. A. Steen, M. Forsman. Paraplegia after thoracotomy—not caused by the epidural catheter. Acta Anaesthesiol Scand 43:230-232 (1999).

E. E. Awwad, K. R. Smith Jr. MRI of marked dural sac compression by Surgicel in the immediately postoperative period after uncomplicated lumbar laminectomy. J Comput Assist Tomogr 23: 969-975 (1999).

D. H. Alander, E. S. Stauffer Gelfoam-induced acute quadriparesis after cervical decompression and fusion. Spine 20:970-971 (1995).

J. Friedman, T. S. Whitecloud III. Lumbar cauda equina syndrome associated with the use of Gelfoam: case report. Spine 26: E485-E487 (2001).

FDA Public Health Notification: Paralysis from absorbable haemostatic agent. Feb. 4, 2004. http://wvvw.fda.gov/cdrh/safety/040204-haemostatics.html.

B. A. Roeder, K. Kokini, J. E. Stirgis, J. P. Robinson, S. L. Voytik-Harbin, Tensile Mechanical Properties of Three-Dimensional Type I Collagen Extracellular Matrices with Varied Microstructure, Transactions of the ASME, 124: 214-222 (2002).

W. Friess, Collagen—biomaterial for drug delivery. Eur J. Pharma & Biopharma 45: 113-136 (1998).

A. Salehpur et al, Dose dependent response of gamma irradiation on mechanical properties and related biochemical composition of goat bone-patellar tendon-bone allografts. J. Orthop Res 13: 898-906 (1995).

Liu, B. C. et al, The effect of gamma irradiation on injectable human amnion collagen. J Biomed Mater Res 23: 833-844 (1989).

E. M. Noah et al, Impact of sterilisation on the porous design and cell behaviour in collagen sponges prepared for tissue engineering. Biomaterials 23: 2855-2861 (2002).

L. H. H Olde Damink, P. J. Dijkstra, M. J. A van Luyn, P. B. van Wachem, P. Nieuwenhuis, J. Feijen, Influence of ethylene oxide gas treatment on the in vitro degradation behaviour of dermal sheep collagen. J. Biomed. Mater. Res.29 149-155 (1995).

I. L. Mello, M. C. Delpech, F. M. B. Coutinho and F. F. M. Albino-Viscometric Study of High-cis Polybutadiene in Toluene Solution J. Braz. Chem. Soc., Vol. 17, No. 1, 194-199 (2006).

A. Stemberger, H. Grimm, F. Bader, H. D. Rahn, R. Ascher, Local Treatment of Bone and Soft Tissue Infections With Collagen—Gentamycin Sponge, Eur. J. Surg. 163: Supplement 578, 17-26 (1997).

A. Stemberger, J. Scherer, R. Ascherl, M. A. Scherer, F. Lechner, G. Blumel, A biological drug-carrier in soft tissue infections. Progress in Angiology, 275-277 (1988).

1. An implant suitable for delivery of at least one drug, the implant comprising a fibrillar collagen matrix having, as measured in Example 1, a viscosity of greater than 100 mPas, when a collagen dispersion formed from 140 mg of the fibrillar collagen matrix is dispersed in 25 ml of 2 mM HCl at a pH of less than 3.5 and at a temperature of 29.5 to 30.5° C.

2. The implant of claim 1, wherein the implant shows a volume reduction of at least 30%, when 70 mg of the fibrillar collagen matrix is immersed in 50 ml of 0.9% sodium chloride for 10 minutes at 37° C.

3. The implant of claim 2, wherein the volume reduction is at least 50%.

4. The implant of claim 1, wherein a pharmaceutically acceptable amount of at least one drug is dispersed in the fibrillar collagen matrix, in which the at least one drug has at least a localised pharmacological action.

5. The implant of claim 4, wherein the at least one drug is selected from the group consisting of anaesthetics opioid-type analgesics; anti-inflammatory analgesics; chemotherapeutic agents; and anti-microbial agents, pharmaceutically acceptable salts of any thereof, and a combination of any thereof.

6. The implant of claim 4, wherein the at least one drug is selected from the group consisting of amino amide anaesthetics and salts thereof, opioid-type analgesics and salts thereof and non-steroidal anti-inflammatory analgesics, and a mixture thereof.

7. The implant of claim 6, wherein the at least one amino amide anaesthetic is selected from the group consisting of lidocaine, prilocaine, bupivicaine and its enantiomer levobupivacaine, ropivacaine, mepivacaine, dibucaine, including the hydrochloride salts of any thereof, and a mixture of any thereof.

8. The implant of claim 6, wherein the at least one opioid-type analgesics is selected from the group consisting of morphine and its salts; diamorphine and its hydrochloride salt; desomorphine; codeine and its salts; hydrocodone and its bitartrate salt; hydromorphone and its hydrochloride salt; oxycodone; oxymorphone; fentanyl and its related analogues comprising alfentanil, sufentanil, remifentanil, carfentanil and lofentanil; buprenorphine and its hydrochloride salt, tramadol and its hydrochloride and tartrate salts, tapentadol, and a mixture of any thereof.

9. The implant of claim 6, wherein the at least non-steroidal anti-inflammatory drug is selected from the group consisting of diclofenac sodium and potassium salts, ketoprofen and its active enantiomer dexketoprofen, naproxen and its sodium salt, ibuprofen and its sodium salt and its active enantiomer dexibuprofen, meloxicam, piroxicam, indomethacin, acetylsalicylic acid, and a mixture of any thereof.

10. A process for preparing an implant suitable for delivery of at least one drug, the process comprising the steps of
forming a fibrillar collagen matrix from a collagen suspension; and
carrying out a crosslinking step on either the fibrillar collagen matrix or the collagen suspension under conditions such that the fibrillar collagen matrix has, as measured in Example 1, a viscosity of greater than 100 mPas, when a collagen dispersion formed from 140 mg of the fibrillar collagen matrix is dispersed in 25 ml of 2 mM HCl at a pH of less than 3.5 and at a temperature of 29.5 to 30.5° C.

11. The process of claim 10, wherein the crosslinking step is carried out on the fibrillar collagen matrix, before or after incorporation of the at least one drug.

12. The process of claim 10, wherein the crosslinking step is selected from ethylene oxide (EO) sterilisation, electron beam (E-beam) sterilisation, dehydrothermal crosslinking, chemical crosslinking, or a combination thereof.

13. The process of claim 12, wherein the crosslinking step is ethylene oxide (EO) sterilisation, and the ethylene oxide (EO) sterilisation conditions comprise an ethylene oxide (EO) sterilisation time of 6 hours with a chamber pressure of 3.6 to 4.1 bar, a chamber temperature of 30° C. to 40° C. and an ethylene oxide concentration of more than 1300 mg EO/l.

14. The process of claim 12, wherein the crosslinking step is electron beam (E-beam) sterilisation and the electron beam (E-beam) sterilisation is carried out at a radiation dose of at least 15 kGy.

15. The process of claim 12, wherein the crosslinking step is chemical crosslinking using glutaraldehyde, carbodiimides or organic peroxides.

16. The process of claim 15, wherein the crosslinking step is dehydrothermal crosslinking or chemical crosslinking and the implant is sterilised using ethylene oxide (EO) sterilisation, electron beam (E-beam) sterilisation or gamma irradiation.

17. The process of claim 10, wherein a volume reduction of at least 30% is obtained by lyophilising the collagen suspension at a collagen concentration of below 25 mg/ml at a pH of less than 4.9 before any drug is added to the collagen suspension, to prepare the implant.

18. The process of claim 17, wherein the volume reduction of at least 30% is obtained by lyophilising the collagen suspension at a collagen concentration of below 25 mg/ml at a pH of between 3.6 and 4.9 before any drug is added to the collagen suspension, to prepare the implant.

19. The process of claim 10, wherein a volume reduction of at least 30% is obtained by lyophilising the collagen suspension at a collagen concentration of less than 11.2 mg/ml, at a pH of about 4.5 before any drug is added to the collagen suspension.

20. The process of claim 17, wherein the volume reduction is at least 50%.

21. (canceled)
